(19) United States
(12) Patent Application Publication
(10) Pub. No.: US 2018/0296559 A1
(43) Pub. Date: Oct. 18, 2018

(54) TREATMENT OF AIRWAY SMOOTH MUSCLE DYSFUNCTION

(71) Applicant: THE UNIVERSITY OF IOWA RESEARCH FOUNDATION, Iowa City, IA (US)

(72) Inventors: David A. STOLTZ, Iowa City, IA (US); Daniel COOK, Iowa City, IA (US)

(73) Assignee: THE UNIVERSITY OF IOWA RESEARCH FOUNDATION, Iowa City, IA (US)

(21) Appl. No.: 15/724,361

(22) Filed: Oct. 4, 2017

Related U.S. Application Data
(60) Provisional application No. 62/404,961, filed on Oct. 6, 2016.

(51) Int. Cl. A61K 31/506; A61K 9/00; A61K 45/06; A61P 11/00

(52) U.S. Cl. CPC A61K 31/506; A61K 9/0053; A61P 11/00; A61K 9/0073; A61K 45/06; A61K 9/0019

(57) ABSTRACT

The present disclosure is directed to compositions and methods for treating airway smooth muscle dysfunction in a subject. The compounds target the tyrosine protein kinase 2 beta. A particular dysfunction is asthma, including cystic fibrosis-induced asthma.

This application claims the benefit of priority to U.S. Provisional Patent Application No. 62/404,961, filed Oct. 6, 2016, the entirety of which is incorporated herein by reference.

### BACKGROUND

### 1. Field of the Disclosure

The present disclosure relates generally to the fields of medicine, cell biology, and pharmaceutical chemistry. More particular, the disclosure relates to airway smooth muscle dysfunction methods of treating the same using compounds that inhibit PYK2 and/or ALK activity.

2. Background

Cystic fibrosis (CF) airway disease is characterized by progressive airflow obstruction. Subsets of individuals with CF also develop airway hyper-responsiveness to inhaled cholinergic is agonists (Weinberger, 2002 and Mitchell et al., 1978) and reversibility of airflow limitation in response to bronchodilators (van Haren et al., 1991 and van Haren et al., 1992). The presence of bronchial hyper-responsiveness and airway obstruction suggest a possible shared etiology of disease between CF and other diseases of airway narrowing such as asthma or chronic obstructive pulmonary disease (COPD) where airway smooth muscle dysfunction is thought to contribute to the disease processes. Recently, our lab has discovered a novel role for cystic fibrosis transmembrane conductance regulator (CFTR), the chloride and bicarbonate channel mutated in human CF disease, in airway smooth muscle function (Cook et al., 2016).

Many cells and tissues are involved in airway narrowing that are regulated by calcium dynamics (Berridge et al., 2000). Of these, airway smooth muscle is involved in airway contractility, a characteristic trait that is assessed clinically in individuals with CF by bronchodilator responses. How intracellular calcium dynamics in airway smooth muscle contributes to airway hyper-responsiveness and remodeling in diseases of airway narrowing, such as CF, remains to be understood. Because calcium can act through a process known as excitation-transcription coupling to modulate signal transduction pathways and subsequently regulate the process of gene transcription, a better understanding of ASM transcriptome alterations in states of airway hyper-responsiveness is needed to provide mechanistic insights for improving airway narrowing and airflow obstruction therapy. Several studies have been conducted to identify transcriptional changes in states of airway hyper-responsiveness (Yick et al., 2014, Pascoe et al., 2015 and Woodruff, 2008), however these prior studies have been limited by the contributions of other tissue types (primarily the immune system) in the development of airway hyper-responsiveness.

### SUMMARY

Thus, in accordance with the present disclosure, there is provided a method of treating airway smooth muscle dysfunction in a subject comprising administering to said subject an agent selected from a compound having the structure:

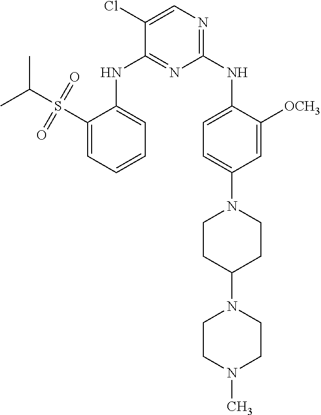 [SMILES: COc1cc(N2CCC(N3CCN(C)CC3)CC2)ccc1Nc1ncc(Cl)c(Nc2ccccc2S(=O)(=O)C(C)C)n1]

or a derivative thereof, or Ceretinib or a derivative thereof. The smooth muscle dysfunction may be airway narrowing, such as asthma or chronic obstructive pulmonary disease, such as cystic fibrosis-induced asthma. Administering may comprise systemic administration, such as oral administration or intravenous administration. Administering may comprise loco-regional administration, such as inhalation administration. The subject may be a human, or a non-human mammal or a pig.

The method may further comprise treating said subject with a second therapy that improves airway function. The second therapy may a steroid-based therapy, a leukotriene is modifying therapy, a bronchodilatory therapy or a beta-agonist therapy. The second therapy may be administered at the same time as said agent, or before or after said compound. The agent may be administered more than once. The agent may administered daily, every other day, every third day, bi-weekly, every fourth day, weekly, every two weeks, or monthly, and/or on a chronic basis.

Also provided is a method of treating smooth muscle dysfunction in a subject comprising administering to said subject an agent that inhibits tyrosine protein kinase 2 beta activity, wherein the smooth muscle dysfunction is not asthma. Administering may comprise systemic administration, including oral administration or intravenous administration, or may comprise loco-regional administration. The subject may be a human or a non-human mammal, such as a pig. The agent may be administered more than once, may be administered daily, every other day, every third day, bi-weekly, every fourth day, weekly, every two weeks, or monthly, and/or may to be administered on a chronic basis. The smooth muscle dysfunction may be selected from the group consisting of vasospastic disorders, pulmonary hypertension, bladder and prostate dysfunction, chronic cramps, migraines, gastrointestinal and esophageal motility disorders, and angina. The agent may be selected from compound having the formula:

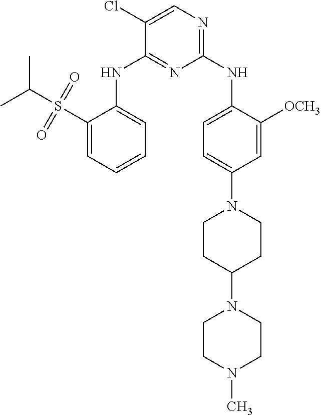 [SMILES: COc1cc(N2CCC(N3CCN(C)CC3)CC2)ccc1Nc1ncc(Cl)c(Nc2ccccc2S(=O)(=O)C(C)C)n1]

or a derivative thereof, or Certinib, or a derivative thereof.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1. NVP-TAE684 decreases airway contraction in porcine precision cut lung slices. Percent airway lumen area contraction in response to methacholine in porcine precision cut lung slices pretreated with control vehicle (DMSO, black circles) or NVP-TAE684 (grey circles), normalized to maximal contractile response (n=6 animals per group). Data are shown as mean values with SEM and are fitted with a four-parameter logistic regression algorithm (solid line).

FIG. 2. NVP-TAE684 decreases airway smooth muscle force generation. Wild-type airway smooth muscle strip isometric force generation following acetylecholine treatment in muscle strips pretreated with control vehicle (DMSO, black circles) or NVP-TAE684 (grey circles) (n=5 animals per group). Data are shown as mean values with SEM and are fitted with a four-parameter logistic regression algorithm (solid line).

FIG. 3. In vivo treatment with NVP-TAE684 decreases cholinergic induced airway hyper-responsiveness. Mice were pretreated with control vehicle (DMSO, black circles) or NVP-TAE684 (grey circles) for 3 days (n=10 animals per group). Airway resistance following methacholine treatment was then determined. Data are shown as mean values with SEM and are fitted with a four-parameter logistic regression algorithm (solid line).

FIG. 4. Recombinant human PYK2 was treated with either DMSO or 10, 25, or 100 μM ceritinib. PYK2 activity was measured by using an ADP-based phosphatase-coupled kinase assay. ADP from PYK2 activity releases an inorganic phosphate via a coupling enzyme and is detected using malachite green reagent. The amount of inorganic phosphate released is proportional to the amount of ADP generated during the kinase reaction. Two concentrations of PYK2 (0.5 and 2 μg/ml) were tested.

### DESCRIPTION OF ILLUSTRATIVE EMBODIMENTS

Abnormal airway smooth muscle function is thought to contribute to airway hyper-reactivity in diseases such as asthma. In cystic fibrosis (CF), airway hyper-responsiveness has been frequently reported and suggests that an altered airway smooth muscle physiology may contribute to CF airway disease. The inventors previously showed that the newborn CFTR^{-/-} pig displays airway smooth muscle abnormalities prior to the onset of inflammation or infection including increased basal tone, increased bronchodilator response, and decreased calcium reuptake. The inventors used the CF pig airway smooth muscle transcriptome to identify potential airway smooth muscle therapeutics for CF and other airway narrowing diseases such as asthma. Total RNA sequencing of newborn non-CF and CF airway smooth muscle revealed differential and significant changes in muscle contraction related genes, ontologies, and pathways. High-throughput ELISA protein comparisons between non-CF and CF airway smooth muscle cells revealed complimentary pathway dysregulation. The genomic and proteomic is signatures were used to perform pathway analysis. Large-scale changes in transcript, protein abundance, and phosphorylation status were significantly different in PI3K/AKT and MAPK controlled pathways in CF samples. PYK2, a calcium-sensitive tyrosine kinase known to be an upstream regulator of both P13K/AKT and MAPK pathways, showed increased phosphorylation status suggesting increased activity. The inventors then used a small molecule inhibitor of PYK2 and ALK, NVP-TAE684, to test whether blocking PYK2 activation could alter airway contractility. Porcine precision cut lung slices pretreated with NVP-TAE684 demonstrated decreased airway narrowing in response to cholinergic stimulation compared to controls. Finally, they demonstrated that pretreatment with NVP-TAE684 reduced methacholine induced airway hyper-responsiveness in vivo using a mouse model. Collectively, these data identified a novel compound and pathway that can modulate airway smooth muscle function and led to the discovery of novel therapeutics for diseases of airflow obstruction.

### I. AIRWAY SMOOTH MUSCLE

A. Smooth Muscle Biology

Smooth muscle is an involuntary non-striated muscle. It is divided into two subgroups;

the single-unit (unitary) and multiunit smooth muscle. Within single-unit cells, the whole bundle or sheet contracts as a syncytium (i.e., a multinucleate mass of cytoplasm that is not separated into cells). Multiunit smooth muscle tissues innervate individual cells; as such, they allow for fine control and gradual responses, much like motor unit recruitment in skeletal muscle.

Smooth muscle is found within the walls of blood vessels (such smooth muscle specifically being termed vascular smooth muscle) such as in the tunica media layer of large (aorta) and small arteries, arterioles and veins. Smooth muscle is also found in lymphatic vessels, the urinary bladder, uterus (termed uterine smooth muscle), male and female reproductive tracts, gastrointestinal tract, respiratory tract, arrector pili of skin, the ciliary muscle, and iris of the eye. The structure and function is basically the same in smooth muscle cells in different organs, but the inducing stimuli differ substantially, in order to perform individual effects in the body at individual times. In addition, the glomeruli of the kidneys contain smooth muscle-like cells called mesangial cells.

A smooth muscle is excited by external stimuli, which causes contraction. Smooth muscle may contract spontaneously (via ionic channel dynamics) or as in the gut special is pacemakers cells interstitial cells of Cajal produce rhythmic contractions. Also, contraction, as well as relaxation, can be induced by a number of physiochemical agents (e.g., hormones, drugs, neurotransmitters—particularly from the autonomic nervous system). Smooth muscle in various regions of the vascular tree, the airway and lungs, kidneys and vagina is different in their expression of ionic channels, hormone receptors, cell-signaling pathways, and other proteins that determine function.

For instance, blood vessels in skin, gastrointestinal system, kidney and brain respond to norepinephrine and epinephrine (from sympathetic stimulation or the adrenal medulla) by producing vasoconstriction (this response is mediated through alpha-1 adrenergic receptors. However, blood vessels within skeletal muscle and cardiac muscle respond to these catecholamines producing vasodilation because the smooth muscle possess beta-adrenergic receptors. So there is a difference in the distribution of the various adrenergic receptors that explains the difference in why blood vessels from different areas respond to the same agent norepinephrine/epinephrine differently as well as differences due to varying amounts of these catecholamines that are released and sensitivities of various receptors to concentrations.

Generally, arterial smooth muscle responds to carbon dioxide by producing vasodilation, and responds to oxygen by producing vasoconstriction. Pulmonary blood vessels within the lung are unique as they vasodilate to high oxygen tension and vasoconstrict when it falls. Bronchiole, smooth muscle that line the airways of the lung, respond to high carbon dioxide producing vasodilation and vasoconstrict when carbon dioxide is low. These responses to carbon dioxide and oxygen by pulmonary blood vessels and bronchiole airway smooth muscle aid in matching perfusion and ventilation within the lungs. Further different smooth muscle tissues display extremes of abundant to little sarcoplasmic reticulum so excitation-contraction coupling varies with its dependence on intracellular or extracellular calcium.

Recent research indicates that sphingosine-1-phosphate (S1P) signaling is an important regulator of vascular smooth muscle contraction. When transmural pressure increases, to sphingosine kinase 1 phosphorylates sphingosine to S1P, which binds to the S1P2 receptor in plasma membrane of cells. This leads to a transient increase in intracellular calcium, and activates Rac and Rhoa signaling pathways. Collectively, these serve to increase MLCK activity and decrease MLCP activity, promoting muscle contraction. This allows arterioles to increase resistance in response to increased blood pressure and thus maintain constant blood flow. The is Rhoa and Rac portion of the signaling pathway provides a calcium-independent way to regulate resistance artery tone.

To maintain organ dimensions against force, cells are fastened to one another by adherens junctions. As a consequence, cells are mechanically coupled to one another such that contraction of one cell invokes some degree of contraction in an adjoining cell. Gap junctions couple adjacent cells chemically and electrically, facilitating the spread of chemicals (e.g., calcium) or action potentials between smooth muscle cells. Single unit smooth muscle displays numerous gap junctions and these tissues often organize into sheets or bundles which contract in bulk.

Smooth muscle contraction is caused by the sliding of myosin and actin filaments (a sliding filament mechanism) over each other. The energy for this to happen is provided by the hydrolysis of ATP. Myosin functions as an ATPase utilizing ATP to produce a molecular conformational change of part of the myosin and produces movement. Movement of the filaments over each other happens when the globular heads protruding from myosin filaments attach and interact with actin filaments to form crossbridges. The myosin heads tilt and drag along the actin filament a small distance (10-12 nm). The heads then release the actin filament and then changes angle to relocate to another site on the actin filament a further distance (10-12 nm) away. They can then re-bind to the actin molecule and drag it along further. This process is called crossbridge cycling and is the same for all muscles (see muscle contraction). Unlike cardiac and skeletal muscle, smooth muscle does not contain the calcium-binding protein troponin. Contraction is initiated by a calcium-regulated phosphorylation of myosin, rather than a calcium-activated troponin system.

Crossbridge cycling causes contraction of myosin and actin complexes, in turn causing increased tension along the entire chains of tensile structures, ultimately resulting in contraction of the entire smooth muscle tissue.

Smooth muscle may contract phasically with rapid contraction and relaxation, or tonically with slow and sustained contraction. The reproductive, digestive, respiratory, and urinary tracts, skin, eye, and vasculature all contain this tonic muscle type. This type of smooth muscle can maintain force for prolonged time with only little energy utilization. There are differences in the myosin heavy and light chains that also correlate with these differences in contractile patterns and kinetics of contraction between tonic and phasic smooth muscle.

Crossbridge cycling cannot occur until the myosin heads have been activated to allow crossbridges to form. When the light chains are phosphorylated, they become active and will allow contraction to occur. The enzyme that phosphorylates the light chains is called myosin light-chain kinase (MLCK), also called MLC₂₀ kinase. In order to control contraction, MLCK will work only when the muscle is stimulated to contract. Stimulation will increase the intracellular concentration of calcium ions. These bind to a molecule called calmodulin, and form a calcium-calmodulin complex. It is this complex that will bind to MLCK to activate it, allowing the chain of reactions for contraction to occur.

Activation consists of phosphorylation of a serine on position 19 (Ser19) on the MLC₂₀ light chain, which causes a conformational change that increases the angle in the neck domain of the myosin heavy chain, which corresponds to the part of the cross-bridge cycle where the myosin head is unattached to the actin filament and relocates to another site on it. After attachment of the myosin head to the actin filament, this serine phosphorylation also activates the ATPase activity of the myosin head region to provide the energy to fuel the subsequent contraction. Phosphorylation of a threonine on position 18 (Thr18) on MLC20 is also possible and may further increase the ATPase activity of the myosin complex. Phosphorylation of the MLC₂₀ myosin light chains correlates well with the shortening velocity of smooth muscle. During this period there is a rapid burst of energy utilization as measured by oxygen consumption. Within a few minutes of initiation the calcium level markedly decrease, MLC₂₀ myosin light chains phosphorylation decreases, and energy utilization decreases and the muscle can relax. Still, smooth muscle has the ability of sustained maintenance of force in this situation as well. This sustained phase has been attributed to certain myosin crossbridges, termed latch-bridges, that are cycling very slowly, notably slowing the progression to the cycle stage whereby dephosphorylated myosin detaches from the actin, thereby maintaining the force at low energy costs. This phenomenon is of great value especially for tonically active smooth muscle.

Isolated preparations of vascular and visceral smooth muscle contract with depolarizing high potassium balanced saline generating a certain amount of contractile force. The same preparation stimulated in normal balanced saline with an agonist such as endothelin or serotonin will generate more contractile force. This increase in force is termed calcium sensitization. The myosin light chain phosphatase is inhibited to increase the gain or sensitivity of myosin light chain kinase to calcium. There are number of cell signalling pathways believed to regulate this is decrease in myosin light chain phosphatase: a RhoA-Rock kinase pathway, a Protein kinase C-Protein kinase C potentiation inhibitor protein 17 (CPI-17) pathway, telokin, and a Zip kinase pathway. Further Rock kinase and Zip kinase have been implicated to directly phosphorylate the 20kd myosin light chains.

Other cell signaling pathways and protein kinases (Protein kinase C, Rho kinase, Zip kinase, Focal adhesion kinases) have been implicated as well and actin polymerization dynamics plays a role in force maintenance. While myosin light chain phosphorylation correlates well with shortening velocity, other cell signaling pathways have been implicated in the development of force and maintenance of force. Notably the phosphorylation of specific tyrosine residues on the focal adhesion adapter protein-paxillin by specific tyrosine kinases has been demonstrated to be essential to force development and maintenance. For example, cyclic nucleotides can relax arterial smooth muscle without reductions in crossbridge phosphorylation, a process termed force suppression. This process is mediated by the phosphorylation of the small heat shock protein, hsp20, and may prevent phosphorylated myosin heads from interacting with actin.

The phosphorylation of the light chains by MLCK is countered by a myosin light-chain phosphatase, which dephosphorylates the MLC₂₀ myosin light chains and thereby inhibits contraction. Other signaling pathways have also been implicated in the regulation actin and myosin dynamics. In general, the relaxation of smooth muscle is by cell-signaling pathways that increase the myosin phosphatase activity, decrease the intracellular calcium levels, hyperpolarize the smooth muscle, and/or regulate actin and myosin muscle can be mediated by the endothelium-derived relaxing factor-nitric oxide, endothelial derived hyperpolarizing factor (either an endogenous cannabinoid, cytochrome P450 metabolite, or hydrogen peroxide), or prostacyclin (PGI2). Nitric oxide and PGI2 stimulate soluble guanylate cyclase and membrane bound adenylate cyclase, respectively. The cyclic nucleotides (cGMP and cAMP) produced by these cyclases activate Protein Kinase G and Protein Kinase A and phosphorylate a number of proteins. The phosphorylation events lead to a decrease in intracellular calcium (inhibit L type Calcium channels, inhibits IP3 receptor channels, stimulates sarcoplasmic reticulum Calcium pump ATPase), a decrease in the 20kd myosin light chain phosphorylation by altering calcium sensitization and increasing myosin light chain phosphatase activity, a stimulation of calcium sensitive potassium channels which hyperpolarize the cell, and the phosphorylation of amino acid residue serine 16 on the small heat shock protein (hsp20)by Protein Kinases A and G. The is phosphorylation of hsp20 appears to alter actin and focal adhesion dynamics and actin-myosin interaction, and recent evidence indicates that hsp20 binding to 14-3-3 protein is involved in this process. An alternative hypothesis is that phosphorylated Hsp20 may also alter the affinity of phosphorylated myosin with actin and inhibit contractility by interfering with crossbridge formation. The endothelium derived hyperpolarizing factor stimulates calcium sensitive potassium channels and/or ATP sensitive potassium channels and stimulate potassium efflux which hyperpolarizes the cell and produces relaxation.

B. Airway Smooth Muscle Structure and Function

The active effector of airway reactivity is airway smooth muscle (ASM), located in the wall of the airways, and is present in the bronchial tree of most vertebrates studied including the pig, ox, cat, dog, rabbit and guinea-pig. ASM has many possible functions including control of airflow distribution, alterations in the size of the dead space, assisting contraction of the lung, expelling air or foreign material, controlling airway caliber during different phases of respiration and allowing variations in the compliance of the airway wall to match functions such as tidal breathing, deep breathing, and coughing or forced expiration. Airway smooth muscle was first described in detail in 1822, and this and other early descriptive studies showed that airway smooth muscle is present in both the central and peripheral airways, relatively more prominent in the peripheral airways, more transverse in central airways and slightly more longitudinal in peripheral airways, and arranged in a helical or geodesic pattern which is more apparent in peripheral airways and when the lung is fully inflated. The contractile state of ASM is modulated by a variety of extracellular agonists acting on specific receptors located in the plasma membrane of ASM cells (ASMCs). Stimulation of these receptors activates a cascade of intracellular events that lead to ASMC contraction or relaxation.

C. ASM-Related Diseases

1. Asthma

Asthma is a common long term inflammatory disease of the airways of the lungs. It is characterized by variable and recurring symptoms, reversible airflow obstruction, and bronchospasm. Symptoms include episodes of wheezing, coughing, chest tightness, and shortness of breath. These episodes may occur a few times a day or a few times per week. is Depending on the person they may become worse at night or with exercise.

Asthma is thought to be caused by a combination of genetic and environmental factors. Environmental factors include exposure to air pollution and allergens. Other potential triggers include medications such as aspirin and beta blockers. Diagnosis is usually based on the pattern of symptoms, response to therapy over time, and spirometry. Asthma is classified according to the frequency of symptoms, forced expiratory volume in one second (FEV1), and peak expiratory flow rate. It may also be classified as atopic or non-atopic where atopy refers to a predisposition toward developing a type 1 hypersensitivity reaction.

There is no cure for asthma. Symptoms can be prevented by avoiding triggers, such as allergens and irritants, and by the use of inhaled corticosteroids. Long-acting beta agonists (LABA) or antileukotriene agents may be used in addition to inhaled corticosteroids if asthma symptoms remain uncontrolled. Treatment of rapidly worsening symptoms is usually with an inhaled short-acting beta-2 agonist such as salbutamol and corticosteroids taken by mouth. In very severe cases, intravenous corticosteroids, magnesium sulfate, and hospitalization may be required.

In 2013, 242 million people globally had asthma up from 183 million in 1990. It caused about 489,000 deaths in 2013, most of which occurred in the developing world. It often begins in childhood. The rates of asthma have increased significantly since the 1960s.

Asthma is characterized by recurrent episodes of wheezing, shortness of breath, chest tightness, and coughing. Sputum may be produced from the lung by coughing but is often hard to bring up. During recovery from an attack, it may appear pus-like due to high levels of white blood cells called eosinophils. Symptoms are usually worse at night and in the early morning or in response to exercise or cold air. Some people with asthma rarely experience symptoms, usually in response to triggers, whereas others may have marked and persistent symptoms. Associated conditions. A number of other health conditions occur more frequently in those with asthma, including gastro-esophageal reflux disease (GERD), rhinosinusitis, and obstructive sleep apnea. Psychological disorders are also more common, with anxiety disorders occurring in between 16-52% and mood disorders in 14-41%. However, it is not known if asthma causes psychological problems or if psychological problems lead to asthma. Those with asthma, especially if it is poorly controlled, are at high risk for radiocontrast reactions.

Causes. Asthma is caused by a combination of complex and incompletely understood environmental and genetic interactions. These factors influence both its severity and its responsiveness to treatment. It is believed that the recent increased rates of asthma are due to changing epigenetics (heritable factors other than those related to the DNA sequence) and a changing living environment. Onset before age 12 is more likely due to genetic influence, while onset after 12 is more likely due to environmental influence.

Environmental. Many environmental factors have been associated with asthma's development and exacerbation including allergens, air pollution, and other environmental chemicals. Smoking during pregnancy and after delivery is associated with a greater risk of asthma-like symptoms. Low air quality from factors such as traffic pollution or high ozone levels, has been associated with both asthma development and increased asthma severity. Over half of cases in children in the United States occur in areas with air quality below EPA standards. Exposure to indoor volatile organic compounds may be a trigger for asthma; formaldehyde exposure, for example, has a positive association. Also, phthalates in certain types of PVC are associated with asthma in children and adults.

There is an association between acetaminophen (paracetamol) use and asthma. The majority of the evidence does not, however, support a causal role. A 2014 review found that the association disappeared when respiratory infections were taken into account. Use by a mother during pregnancy is also associated with an increased risk as is psychological stress during pregnancy.

Asthma is associated with exposure to indoor allergens. Common indoor allergens include dust mites, cockroaches, animal dander, and mold. Efforts to decrease dust mites have been found to be ineffective on symptoms in sensitized subjects. Certain viral respiratory infections, such as respiratory syncytial virus and rhinovirus, may increase the risk of developing asthma when acquired as young children. Certain other infections, however, may decrease the risk.

Hygiene hypothesis. The hygiene hypothesis attempts to explain the increased rates of asthma worldwide as a direct and unintended result of reduced exposure, during childhood, to non-pathogenic bacteria and viruses. It has been proposed that the reduced exposure to bacteria is and viruses is due, in part, to increased cleanliness and decreased family size in modern societies. Exposure to bacterial endotoxin in early childhood may prevent the development of asthma, but exposure at an older age may provoke bronchoconstriction. Evidence supporting the hygiene hypothesis includes lower rates of asthma on farms and in households with pets.

Use of antibiotics in early life has been linked to the development of asthma. Also, delivery via caesarean section is associated with an increased risk (estimated at 20-80%) of asthma—this increased risk is attributed to the lack of healthy bacterial colonization that the newborn would have acquired from passage through the birth canal. There is a link between asthma and the degree of affluence.

Genetic. Family history is a risk factor for asthma, with many different genes being implicated. If one identical twin is affected, the probability of the other having the disease is approximately 25%. By the end of 2005, 25 genes had been associated with asthma in six or more separate populations, including GSTM1, IL10, CTLA-4, SPINK5, LTC4S, IL4R and ADAM33, among others. Many of these genes are related to the immune system or modulating inflammation. Even among this list of genes supported by highly replicated studies, results have not been consistent among all populations tested. In 2006 over 100 genes were associated with asthma in one genetic association study alone; more continue to be found.

Some genetic variants may only cause asthma when they are combined with specific environmental exposures. An example is a specific single nucleotide polymorphism in the CD14 region and exposure to endotoxin (a bacterial product). Endotoxin exposure can come from several environmental sources including tobacco smoke, dogs, and farms. Risk for asthma, then, is determined by both a person's genetics and the level of endotoxin exposure.

Medical conditions. A triad of atopic eczema, allergic rhinitis and asthma is called atopy. The strongest risk factor for developing asthma is a history of atopic disease; with asthma occurring at a much greater rate in those who have either eczema or hay fever. Asthma has been associated with Churg-Strauss syndrome, an autoimmune disease and vasculitis. Individuals with certain types of urticaria may also experience symptoms of asthma.

There is a correlation between obesity and the risk of asthma with both having increased in recent years. Several factors may be at play including decreased respiratory function due to a buildup of fat and the fact that adipose tissue leads to a pro-inflammatory state.

Beta blocker medications such as propranolol can trigger asthma in those who are is susceptible. Cardioselective beta-blockers, however, appear safe in those with mild or moderate disease. Other medications that can cause problems in asthmatics are angiotensin-converting enzyme inhibitors, aspirin, and NSAIDs.

Exacerbation. Some individuals will have stable asthma for weeks or months and then suddenly develop an episode of acute asthma. Different individuals react to various factors in different ways. Most individuals can develop severe exacerbation from a number of triggering agents.

Home factors that can lead to exacerbation of asthma include dust, animal dander (especially cat and dog hair), cockroach allergens and mold. Perfumes are a common cause of acute attacks in women and children. Both viral and bacterial infections of the upper respiratory tract can worsen the disease. Psychological stress may worsen symptoms—it is thought that stress alters the immune system and thus increases the airway inflammatory response to allergens and irritants.

Pathophysiology. Asthma is the result of chronic inflammation of the conducting zone of the airways (most especially the bronchi and bronchioles), which subsequently results in increased contractability of the surrounding smooth muscles. This among other factors leads to bouts of narrowing of the airway and the classic symptoms of wheezing. The narrowing is typically reversible with or without treatment. Occasionally the airways themselves change. Typical changes in the airways include an increase in eosinophils and thickening of the lamina reticularis. Chronically the airways' smooth muscle may increase in size along with an increase in the numbers of mucous glands. Other cell types involved include: T lymphocytes, macrophages, and neutrophils. There may also be involvement of other components of the immune system including: cytokines, chemokines, histamine, and leukotrienes among others.

Diagnosis. While asthma is a well-recognized condition, there is not one universal agreed upon definition. It is defined by the Global Initiative for Asthma as “a chronic inflammatory disorder of the airways in which many cells and cellular elements play a role. The chronic inflammation is associated with airway hyper-responsiveness that leads to recurrent episodes of wheezing, breathlessness, chest tightness and coughing particularly at night or in the early morning. These episodes are usually associated with widespread but variable airflow obstruction within the lung that is often reversible either spontaneously or with treatment.”

There is currently no precise test for the diagnosis, which is typically based on the pattern of symptoms and response to therapy over time. A diagnosis of asthma should be suspected if there is a history of recurrent wheezing, coughing or difficulty breathing and these symptoms occur or worsen due to exercise, viral infections, allergens or air pollution. Spirometry is then used to confirm the diagnosis. In children under the age of six the diagnosis is more difficult as they are too young for spirometry.

Spirometry is recommended to aid in diagnosis and management. It is the single best test for asthma. If the FEV1 measured by this technique improves more than 12% following administration of a bronchodilator such as salbutamol, this is supportive of the diagnosis. It however may be normal in those with a history of mild asthma, not currently acting up. As caffeine is a bronchodilator in people with asthma, the use of caffeine before a lung function test may interfere with the results. Single-breath diffusing capacity can help differentiate asthma from COPD. It is reasonable to perform spirometry every one or two years to follow how well a person's asthma is controlled.

The methacholine challenge involves the inhalation of increasing concentrations of a substance that causes airway narrowing in those predisposed. If negative it means that a person does not have asthma; if positive, however, it is not specific for the disease.

Other supportive evidence includes: a ≥20% difference in peak expiratory flow rate on at least three days in a week for at least two weeks, a ≥20% improvement of peak flow following treatment with either salbutamol, inhaled corticosteroids or prednisone, or a ≥20% decrease in peak flow following exposure to a trigger. Testing peak expiratory flow is more variable than spirometry, however, and thus not recommended for routine diagnosis. It may be useful for daily self-monitoring in those with moderate to severe disease and for checking the effectiveness of new medications. It may also be helpful in guiding treatment in those with acute exacerbations.

Classification. Asthma is clinically classified according to the frequency of symptoms, forced expiratory volume in one second (FEV₁), and peak expiratory flow rate. Asthma may also be classified as atopic (extrinsic) or non-atopic (intrinsic), based on whether symptoms are precipitated by allergens (atopic) or not (non-atopic). While asthma is classified based on severity, at the moment there is no clear method for classifying different subgroups of asthma beyond this system. Finding ways to identify subgroups that respond well to different types of treatments is a current critical goal of asthma research.

Although asthma is a chronic obstructive condition, it is not considered as a part of chronic obstructive pulmonary disease as this term refers specifically to combinations of disease that are irreversible such as bronchiectasis, chronic bronchitis, and emphysema. Unlike these diseases, the airway obstruction in asthma is usually reversible; however, if left untreated, the chronic inflammation from asthma can lead the lungs to become irreversibly obstructed due to airway remodeling. In contrast to emphysema, asthma affects the bronchi, not the alveoli.

An acute asthma exacerbation is commonly referred to as an asthma attack. The classic symptoms are shortness of breath, wheezing, and chest tightness. The wheezing is most often when breathing out. While these are the primary symptoms of asthma, some people present primarily with coughing, and in severe cases, air motion may be significantly impaired such that no wheezing is heard. In children, chest pain is often present.

Signs which occur during an asthma attack include the use of accessory muscles of respiration (sternocleidomastoid and scalene muscles of the neck), there may be a paradoxical pulse (a pulse that is weaker during inhalation and stronger during exhalation), and over-inflation of the chest. A blue color of the skin and nails may occur from lack of oxygen.

In a mild exacerbation the peak expiratory flow rate (PEFR) is ≥200 L/min or ≥50% of the predicted best. Moderate is defined as between 80 and 200 L/min or 25% and 50% of the predicted best while severe is defined as ≤80 L/min or ≤25% of the predicted best.

Acute severe asthma, previously known as status asthmaticus, is an acute exacerbation of asthma that does not respond to standard treatments of bronchodilators and corticosteroids. Half of cases are due to infections with others caused by allergen, air pollution, or insufficient or inappropriate medication use.

Brittle asthma is a kind of asthma distinguishable by recurrent, severe attacks. Type 1 brittle asthma is a disease with wide peak flow variability, despite intense medication. Type 2 brittle asthma is background well-controlled asthma with sudden severe exacerbations.

Exercise can trigger bronchoconstriction both in people with or without asthma. It occurs in most people with asthma and up to 20% of people without asthma. Exercise-induced bronchoconstriction is common in professional athletes. The highest rates are among cyclists (up to 45%), swimmers, and cross-country skiers. While it may occur with any weather conditions it is more common when it is dry and cold. Inhaled beta2-agonists do not appear to improve athletic performance among those without asthma however oral doses may improve endurance and strength.

Asthma as a result of (or worsened by) workplace exposures, is a commonly reported occupational disease. Many cases however are not reported or recognized as such. It is estimated that 5-25% of asthma cases in adults are work-related. A few hundred different agents have been implicated with the most common being: isocyanates, grain and wood dust, colophony, soldering flux, latex, animals, and aldehydes. The employment associated with the highest risk of problems include: those who spray paint, bakers and those who process food, nurses, chemical workers, those who work with animals, welders, hairdressers and timber workers.

Aspirin-exacerbated respiratory disease, also known as aspirin-induced asthma, affects up to 9% of asthmatics. Reactions may also occur to other NSAIDs. People affected often also have trouble with nasal polyps. In people who are affected low doses paracetamol or COX-2 inhibitors are generally safe.

Alcohol may worsen asthmatic symptoms in up to a third of people. This may be even more common in some ethnic groups such as the Japanese and those with aspirin-induced asthma. Other studies have found improvement in asthmatic symptoms from alcohol.

Nonallergic asthma, also known as intrinsic or nonatopic asthma makes up between 10 and 33% of cases. There is negative skin test to common inhalant allergens and normal serum concentrations of IgE. Often it starts latter and life and women are more commonly affect than men. Usual treatments may not work as well.

Differential diagnosis. Many other conditions can cause symptoms similar to those of asthma. In children, other upper airway diseases such as allergic rhinitis and sinusitis should be considered as well as other causes of airway obstruction including: foreign body aspiration, tracheal stenosis or laryngotracheomalacia, vascular rings, enlarged lymph nodes or neck masses. Bronchiolitis and other viral infections may also produce wheezing. In adults, COPD, congestive heart failure, airway masses, as well as drug-induced coughing due to ACE inhibitors should be considered. In both populations vocal cord dysfunction may present similarly.

Chronic obstructive pulmonary disease can coexist with asthma and can occur as a complication of chronic asthma. After the age of 65, most people with obstructive airway disease will have asthma and COPD. In this setting, COPD can be differentiated by increased airway is neutrophils, abnormally increased wall thickness, and increased smooth muscle in the bronchi.

However, this level of investigation is not performed due to COPD and asthma sharing similar principles of management: corticosteroids, long-acting beta-agonists, and smoking cessation. It closely resembles asthma in symptoms, is correlated with more exposure to cigarette smoke, an older age, less symptom reversibility after bronchodilator administration, and decreased likelihood of family history of atopy.

Prevention. The evidence for the effectiveness of measures to prevent the development of asthma is weak. Some show promise including: limiting smoke exposure both in utero and after delivery, breastfeeding, and increased exposure to daycare or large families but none are well supported enough to be recommended for this indication. Early pet exposure may be useful. Results from exposure to pets at other times are inconclusive and it is only recommended that pets be removed from the home if a person has allergic symptoms to said pet. Dietary restrictions during pregnancy or when breast feeding have not been found to be effective and thus are not recommended. Reducing or eliminating compounds known to sensitive people from the work place may be effective. It is not clear if annual influenza vaccinations effects the risk of exacerbations. Immunization; however, is recommended by the World Health Organization. Smoking bans are effective in decreasing exacerbations of asthma.

Management. While there is no cure for asthma, symptoms can typically be improved. A specific, customized plan for proactively monitoring and managing symptoms should be created. This plan should include the reduction of exposure to allergens, testing to assess the severity of symptoms, and the usage of medications. The treatment plan should be written down and advise adjustments to treatment according to changes in symptoms.

The most effective treatment for asthma is identifying triggers, such as cigarette smoke, pets, or aspirin, and eliminating exposure to them. If trigger avoidance is insufficient, the use of medication is recommended. Pharmaceutical drugs are selected based on, among other things, the severity of illness and the frequency of symptoms. Specific medications for asthma are broadly classified into fast-acting and long-acting categories.

Bronchodilators are recommended for short-term relief of symptoms. In those with occasional attacks, no other medication is needed. If mild persistent disease is present (more than two attacks a week), low-dose inhaled corticosteroids or alternatively, an oral leukotriene antagonist or a mast cell stabilizer is recommended. For those who have daily attacks, a higher is dose of inhaled corticosteroids is used. In a moderate or severe exacerbation, oral corticosteroids are added to these treatments.

Avoidance of triggers is a key component of improving control and preventing attacks. The most common triggers include allergens, smoke (tobacco and other), air pollution, non-selective beta-blockers, and sulfite-containing foods. Cigarette smoking and second-hand smoke (passive smoke) may reduce the effectiveness of medications such as corticosteroids. Laws that limit smoking decrease the number of people hospitalized for asthma. Dust mite control measures, including air filtration, chemicals to kill mites, vacuuming, mattress covers and others methods had no effect on asthma symptoms. Overall, exercise is beneficial in people with stable asthma. Yoga could provide small improvements in quality of life and symptoms in people with asthma.

Medications used to treat asthma are divided into two general classes: quick-relief medications used to treat acute symptoms; and long-term control medications used to prevent further exacerbation.

Short-acting beta₂-adrenoceptor agonists (SABA), such as salbutamol (albuterol USAN) are the first line treatment for asthma symptoms. They are recommended before exercise in those with exercise induced symptoms.

Anticholinergic medications, such as ipratropium bromide, provide additional benefit when used in combination with SABA in those with moderate or severe symptoms. Anticholinergic bronchodilators can also be used if a person cannot tolerate a SABA. If a child requires admission to hospital additional ipratropium does not appear to help over a SABA.

Older, less selective adrenergic agonists, such as inhaled epinephrine, have similar efficacy to SABAs. They are however not recommended due to concerns regarding excessive cardiac stimulation.

Corticosteroids are generally considered the most effective treatment available for long-term control. Inhaled forms such as beclomethasone are usually used except in the case of severe persistent disease, in which oral corticosteroids may be needed. It is usually recommended that inhaled formulations be used once or twice daily, depending on the severity of symptoms.

Long-acting beta-adrenoceptor agonists (LABA) such as salmeterol and formoterol can improve asthma control, at least in adults, when given in combination with inhaled corticosteroids. In children this benefit is uncertain. When used without steroids they increase the risk of severe side-effects and even with corticosteroids they may slightly increase the risk.

Leukotriene receptor antagonists (such as montelukast and zafirlukast) may be used in addition to inhaled corticosteroids, typically also in conjunction with a LABA. Evidence is insufficient to support use in acute exacerbations. In children they appear to be of little benefit when added to inhaled steroids, and the same applies in adolescents and adults. They are useful by themselves. In those under five years of age, they were the preferred add-on therapy after inhaled corticosteroids by the British Thoracic Society in 2009. A similar class of drugs, 5-LOX inhibitors, may be used as an alternative in the chronic treatment of mild to moderate asthma among older children and adults. As of 2013 there is one medication in this family known as zileuton.

Mast cell stabilizers (such as cromolyn sodium) are another non-preferred alternative to corticosteroids.

Medications are typically provided as metered-dose inhalers (MDIs) in combination with an asthma spacer or as a dry powder inhaler. The spacer is a plastic cylinder that mixes the medication with air, making it easier to receive a full dose of the drug. A nebulizer may also be used. Nebulizers and spacers are equally effective in those with mild to moderate symptoms. However, insufficient evidence is available to determine whether a difference exists in those with severe disease.

Long-term use of inhaled corticosteroids at conventional doses carries a minor risk of adverse effects. Risks include the development of cataracts and a mild regression in stature.

When asthma is unresponsive to usual medications, other options are available for both emergency management and prevention of flareups. For example, oxygen is used alleviate hypoxia if saturations fall below 92%. Oral corticosteroid are recommended with five days of prednisone being the same 2 days of dexamethasone. Magnesium sulfate intravenous treatment increases bronchodilation when used in addition to other treatment in moderate severe acute asthma attacks. In adults it results in a reduction of hospital admissions. Heliox, a mixture of helium and oxygen, may also be considered in severe unresponsive cases. Methylxanthines (such as theophylline) were once widely used, but do not add significantly to the effects of inhaled beta-agonists. Their use in acute exacerbations is controversial. The dissociative anesthetic ketamine is theoretically useful if intubation and mechanical ventilation is needed in people who is are approaching respiratory arrest; however, there is no evidence from clinical trials to support this.

For those with severe persistent asthma not controlled by inhaled corticosteroids and LABAs, bronchial thermoplasty may be an option. It involves the delivery of controlled thermal energy to the airway wall during a series of bronchoscopies. While it may increase exacerbation frequency in the first few months it appears to decrease the subsequent rate. Effects beyond one year are unknown. Evidence suggests that sublingual immunotherapy in those with both allergic rhinitis and asthma improve outcomes.

2. Chronic Obstructive Pulmonary Disease/Chronic Bronchitis

Chronic obstructive pulmonary disease (COPD) is a type of obstructive lung disease characterized by long term poor airflow. The main symptoms include shortness of breath and cough with sputum production. COPD typically worsens over time. Eventually walking upstairs or carrying things will be difficult. Chronic bronchitis and emphysema are older terms used for different types of COPD. The term “chronic bronchitis” is still used to define a productive cough that is present for at least three months each year for two years.

Tobacco smoking is the most common cause of COPD, with a number of other factors such as air pollution and genetics playing a smaller role. In the developing world, one of the common sources of air pollution is poorly vented heating and cooking fires. Long-term exposure to these irritants causes an inflammatory response in the lungs resulting in narrowing of the small airways and breakdown of lung tissue. The diagnosis is based on poor airflow as measured by lung function tests. In contrast to asthma, the airflow reduction does not improve much with the use of a bronchodilator.

Most cases of COPD can be prevented by reducing exposure to risk factors. This includes decreasing rates of smoking and improving indoor and outdoor air quality. While treatment can slow worsening there is no cure. COPD treatments include stopping smoking, vaccinations, respiratory rehabilitation, and often inhaled bronchodilators and steroids. Some people may benefit from long-term oxygen therapy or lung transplantation. In those who have periods of acute worsening, increased use of medications and hospitalization may be needed.

As of 2013 COPD affects 329 million people or nearly 5 percent of the global population. is It typically occurs in people over the age of 40. Males and females are affected equally commonly. In 2013 it resulted in 2.9 million deaths, up from 2.4 million deaths in 1990. More than 90% of these deaths occur in the developing world. The number of deaths is projected to increase further because of higher smoking rates and an aging population in many countries. It resulted in an estimated economic cost of $2.1 trillion in 2010.

Symptoms. The most common symptoms of COPD are sputum production, shortness of breath, and a productive cough. These symptoms are present for a prolonged period of time and typically worsen over time. It is unclear if different types of COPD exist. While previously divided into emphysema and chronic bronchitis, emphysema is only a description of lung changes rather than a disease itself, and chronic bronchitis is simply a descriptor of symptoms that may or may not occur with COPD.

A chronic cough is often the first symptom to develop. When it persists for more than three months each year for at least two years, in combination with sputum production and without another explanation, there is by definition chronic bronchitis. This condition can occur before COPD fully develops. The amount of sputum produced can change over hours to days. In some cases, the cough may not be present or may only occur occasionally and may not be productive. Some people with COPD attribute the symptoms to a “smoker's cough”. Sputum may be swallowed or spat out, depending often on social and cultural factors. Vigorous coughing may lead to rib fractures or a brief loss of consciousness. Those with COPD often have a history of “common colds” that last a long time.

Shortness of breath is often the symptom that most bothers people. It is commonly described as: “my breathing requires effort,” “I feel out of breath,” or “I can't get enough air in”. Different terms, however, may be used in different cultures. Typically the shortness of breath is worse on exertion of a prolonged duration and worsens over time. In the advanced stages, it occurs during rest and may be always present. It is a source of both anxiety and a poor quality of life in those with COPD. Many people with more advanced COPD breathe through pursed lips and this action can improve shortness of breath in some.

In COPD, it may take longer to breathe out than to breathe in. Chest tightness may occur but is not common and may be caused by another problem. Those with obstructed airflow may have wheezing or decreased sounds with air entry on examination of the chest with a stethoscope. A barrel chest is a characteristic sign of COPD, but is relatively uncommon. Tripod positioning is may occur as the disease worsens.

Advanced COPD leads to high pressure on the lung arteries, which strains the right ventricle of the heart. This situation is referred to as cor pulmonale, and leads to symptoms of leg swelling and bulging neck veins. COPD is more common than any other lung disease as a cause of cor pulmonale. Cor pulmonale has become less common since the use of supplemental oxygen. COPD often occurs along with a number of other conditions, due in part to shared risk factors. These conditions include ischemic heart disease, high blood pressure, diabetes mellitus, muscle wasting, osteoporosis, lung cancer, anxiety disorder and depression. In those with severe disease, a feeling of always being tired is common. Fingernail clubbing is not specific to COPD and should prompt investigations for an underlying lung cancer.

Exacerbation. An acute exacerbation of COPD is defined as increased shortness of breath, increased sputum production, a change in the color of the sputum from clear to green or yellow, or an increase in cough in someone with COPD. This may present with signs of increased work of breathing such as fast breathing, a fast heart rate, sweating, active use of muscles in the neck, a bluish tinge to the skin, and confusion or combative behavior in very severe exacerbations. Crackles may also be heard over the lungs on examination with a stethoscope.

Cause. The primary cause of COPD is tobacco smoke, with occupational exposure and pollution from indoor fires being significant causes in some countries. Typically these exposures must occur over several decades before symptoms develop. A person's genetic makeup also affects the risk.

The primary risk factor for COPD globally is tobacco smoking. Of those who smoke about 20% will get COPD, and of those who are lifelong smokers about half will get COPD. In the United States and United Kingdom, of those with COPD, 80-95% are either current smokers or previously smoked. The likelihood of developing COPD increases with the total smoke exposure. Additionally, women are more susceptible to the harmful effects of smoke than men. In non-smokers, secondhand smoke is the cause of about 20% of cases. Other types of smoke, such as marijuana, cigar, and water pipe smoke, also confer a risk. Women who smoke during pregnancy may increase the risk of COPD in their child.

Poorly ventilated cooking fires, often fueled by coal or biomass fuels such as wood and animal dung, lead to indoor air pollution and are one of the most common causes of COPD in is developing countries. These fires are a method of cooking and heating for nearly 3 billion people with their health effects being greater among women due to more exposure. They are used as the main source of energy in 80% of homes in India, China and sub-Saharan Africa.

People who live in large cities have a higher rate of COPD compared to people who live in rural areas. While urban air pollution is a contributing factor in exacerbations, its overall role as a cause of COPD is unclear. Areas with poor outdoor air quality, including that from exhaust gas, generally have higher rates of COPD. The overall effect in relation to smoking, however, is believed to be small.

Intense and prolonged exposure to workplace dusts, chemicals and fumes increase the risk of COPD in both smokers and nonsmokers. Workplace exposures are believed to be the cause in 10-20% of cases. In the United States they are believed to be related to more than 30% of cases among those who have never smoked and probably represent a greater risk in countries without sufficient regulations.

A number of industries and sources have been implicated, including high levels of dust in coal mining, gold mining, and the cotton textile industry, occupations involving cadmium and isocyanates, and fumes from welding. Working in agriculture is also a risk. In some professions the risks have been estimated as equivalent to that of one half to two packs of cigarettes a day. Silica dust exposure can also lead to COPD, with the risk unrelated to that for silicosis. The negative effects of dust exposure and cigarette smoke exposure appear to be additive or possibly more than additive.

Genetics play a role in the development of COPD. It is more common among relatives of those with COPD who smoke than unrelated smokers. Currently, the only clearly inherited risk factor is alpha 1-antitrypsin deficiency (AAT). This risk is particularly high if someone deficient in alpha 1-antitrypsin also smokes. It is responsible for about 1-5% of cases and the condition is present in about 3-4 in 10,000 people. Other genetic factors are being investigated, of which there are likely to be many.

A number of other factors are less closely linked to COPD. The risk is greater in those who are poor, although it is not clear if this is due to poverty itself or other risk factors associated with poverty, such as air pollution and malnutrition. There is tentative evidence that those with asthma and airway hyperreactivity are at increased risk of COPD. Birth factors such as low birth weight may also play a role as do a number of infectious diseases including HIV/AIDS and is tuberculosis. Respiratory infections such as pneumonia do not appear to increase the risk of COPD, at least in adults.

An acute exacerbation (a sudden worsening of symptoms) is commonly triggered by infection or environmental pollutants, or sometimes by other factors such as improper use of medications. Infections appear to be the cause of 50 to 75% of cases, with bacteria in 25%, viruses in 25%, and both in 25%. Environmental pollutants include both poor indoor and outdoor air quality. Exposure to personal smoke and secondhand smoke increases the risk. Cold temperature may also play a role, with exacerbations occurring more commonly in winter. Those with more severe underlying disease have more frequent exacerbations: in mild disease 1.8 per year, moderate 2 to 3 per year, and severe 3.4 per year. Those with many exacerbations have a faster rate of deterioration of their lung function. Pulmonary emboli (blood clots in the lungs) can worsen symptoms in those with pre-existing COPD.

Pathophysiology. COPD is a type of obstructive lung disease in which chronic incompletely reversible poor airflow (airflow limitation) and inability to breathe out fully (air trapping) exist. The poor airflow is the result of breakdown of lung tissue (known as emphysema) and small airways disease (known as obstructive bronchiolitis). The relative contributions of these two factors vary between people. Severe destruction of small airways can lead to the formation of large air pockets—known as bullae—that replace lung tissue. This form of disease is called bullous emphysema.

COPD develops as a significant and chronic inflammatory response to inhaled irritants. Chronic bacterial infections may also add to this inflammatory state. The inflammatory cells involved include neutrophil granulocytes and macrophages, two types of white blood cell. Those who smoke additionally have Tc1 lymphocyte involvement and some people with COPD have eosinophil involvement similar to that in asthma. Part of this cell response is brought on by inflammatory mediators such as chemotactic factors. Other processes involved with lung damage include oxidative stress produced by high concentrations of free radicals in tobacco smoke and released by inflammatory cells, and breakdown of the connective tissue of the lungs by proteases that are insufficiently inhibited by protease inhibitors. The destruction of the connective tissue of the lungs is what leads to emphysema, which then contributes to the poor airflow and, finally, poor absorption and release of respiratory gases. General muscle wasting that often occurs in COPD may be partly due to inflammatory mediators released by the lungs into the blood. is Narrowing of the airways occurs due to inflammation and scarring within them. This contributes to the inability to breathe out fully. The greatest reduction in air flow occurs when breathing out, as the pressure in the chest is compressing the airways at this time. This can result in more air from the previous breath remaining within the lungs when the next breath is started, resulting in an increase in the total volume of air in the lungs at any given time, a process called hyperinflation or air trapping. Hyperinflation from exercise is linked to shortness of breath in COPD, as it is less comfortable to breathe in when the lungs are already partly full. Hyperinflation may also worsen during an exacerbation. Some also have a degree of airway hyperresponsiveness to irritants similar to those found in asthma.

Low oxygen levels and, eventually, high carbon dioxide levels in the blood can occur from poor gas exchange due to decreased ventilation from airway obstruction, hyperinflation and a reduced desire to breathe. During exacerbations, airway inflammation is also increased, resulting in increased hyperinflation, reduced expiratory airflow and worsening of gas transfer. This can also lead to insufficient ventilation and, eventually, low blood oxygen levels. Low oxygen levels, if present for a prolonged period, can result in narrowing of the arteries in the lungs, while emphysema leads to breakdown of capillaries in the lungs. Both these changes result in increased blood pressure in the pulmonary arteries, which may cause cor pulmonale.

Diagnosis. The diagnosis of COPD should be considered in anyone over the age of 35 to 40 who has shortness of breath, a chronic cough, sputum production, or frequent winter colds and a history of exposure to risk factors for the disease. Spirometry is then used to confirm the diagnosis. Screening those without symptoms is not recommended.

Spirometry measures the amount of airflow obstruction present and is generally carried out after the use of a bronchodilator, a medication to open up the airways. Two main components are measured to make the diagnosis: the forced expiratory volume in one second (FEV1), which is the greatest volume of air that can be breathed out in the first second of a breath, and the forced vital capacity (FVC), which is the greatest volume of air that can be breathed out in a single large breath. Normally, 75-80% of the FVC comes out in the first second and a FEV₁/FVC ratio of less than 70% in someone with symptoms of COPD defines a person as having the disease. Based on these measurements, spirometry would lead to over-diagnosis of COPD in the elderly. The National Institute for Health and Care Excellence criteria additionally require a FEV₁ of less than 80% of predicted.

Evidence for using spirometry among those without symptoms in an effort to diagnose the condition earlier is of uncertain effect and is therefore currently not recommended. A peak expiratory flow (the maximum speed of expiration), commonly used in asthma, is not sufficient for the diagnosis of COPD.

There are a number of methods to determine how much COPD is affecting a given individual. The modified British Medical Research Council questionnaire (mMRC) or the COPD assessment test (CAT) are simple questionnaires that may be used to determine the severity of symptoms. Scores on CAT range from 0-40 with the higher the score, the more severe the disease. Spirometry may help to determine the severity of airflow limitation. This is typically based on the FEV₁ expressed as a percentage of the predicted “normal” for the person's age, gender, height and weight. Both the American and European guidelines recommended partly basing treatment recommendations on the FEV₁. The GOLD guidelines suggest dividing people into four categories based on symptoms assessment and airflow limitation. Weight loss and muscle weakness, as well as the presence of other diseases, should also be taken into account.

A chest X-ray and complete blood count may be useful to exclude other conditions at the time of diagnosis. Characteristic signs on X-ray are overexpanded lungs, a flattened diaphragm, increased retrosternal airspace, and bullae while it can help exclude other lung diseases, such as pneumonia, pulmonary edema or a pneumothorax. A high-resolution computed tomography scan of the chest may show the distribution of emphysema throughout the lungs and can also be useful to exclude other lung diseases. Unless surgery is planned, however, this rarely affects management. An analysis of arterial blood is used to determine the need for oxygen; this is recommended in those with an FEV₁ less than 35% predicted, those with a peripheral oxygen saturation of less than 92% and those with symptoms of congestive heart failure. In areas of the world where alpha-1 antitrypsin deficiency is common, people with COPD (particularly those below the age of 45 and with emphysema affecting the lower parts of the lungs) should be considered for testing.

Differential diagnosis. COPD may need to be differentiated from other causes of shortness of breath such as congestive heart failure, pulmonary embolism, pneumonia or pneumothorax. Many people with COPD mistakenly think they have asthma. The distinction between asthma and COPD is made on the basis of the symptoms, smoking history, and whether airflow limitation is reversible with bronchodilators at spirometry. Tuberculosis may also present with a chronic cough and should be considered in locations where it is common. Less common conditions that may present similarly include bronchopulmonary dysplasia and obliterative bronchiolitis. Chronic bronchitis may occur with normal airflow and in this situation it is not classified as COPD.

Prevention. Most cases of COPD are potentially preventable through decreasing exposure to smoke and improving air quality. Annual influenza vaccinations in those with COPD reduce exacerbations, hospitalizations and death. Pneumococcal vaccination may also be beneficial.

Keeping people from starting smoking is a key aspect of preventing COPD. The policies of governments, public health agencies, and anti-smoking organizations can reduce smoking rates by discouraging people from starting and encouraging people to stop smoking. Smoking bans in public areas and places of work are important measures to decrease exposure to secondhand smoke and while many places have instituted bans more are recommended.

In those who smoke, stopping smoking is the only measure shown to slow down the worsening of COPD. Even at a late stage of the disease, it can reduce the rate of worsening lung function and delay the onset of disability and death. Smoking cessation starts with the decision to stop smoking, leading to an attempt at quitting. Often several attempts are required before long-term abstinence is achieved. Attempts over 5 years lead to success in nearly 40% of people.

Some smokers can achieve long-term smoking cessation through willpower alone. Smoking, however, is highly addictive, and many smokers need further support. The chance of quitting is improved with social support, engagement in a smoking cessation program and the use of medications such as nicotine replacement therapy, bupropion or varenicline.

A number of measures have been taken to reduce the likelihood that workers in at-risk industries—such as coal mining, construction and stonemasonry—will develop COPD. Examples of these measures include: the creation of public policy, education of workers and management about the risks, promoting smoking cessation, checking workers for early signs of COPD, use of respirators, and dust control. Effective dust control can be achieved by improving ventilation, using water sprays and by using mining techniques that minimize dust generation. If a worker develops COPD, further lung damage can be reduced by avoiding ongoing dust exposure, for example by changing the work role.

Both indoor and outdoor air quality can be improved, which may prevent COPD or slow the worsening of existing disease. This may be achieved by public policy efforts, cultural changes, and personal involvement.

A number of developed countries have successfully improved outdoor air quality through regulations. This has resulted in improvements in the lung function of their populations. Those with COPD may experience fewer symptoms if they stay indoors on days when outdoor air quality is poor.

One key effort is to reduce exposure to smoke from cooking and heating fuels through improved ventilation of homes and better stoves and chimneys. Proper stoves may improve indoor air quality by 85%. Using alternative energy sources such as solar cooking and electrical heating is also effective. Using fuels such as kerosene or coal might be less bad than traditional biomass such as wood or dung.

Management. There is no known cure for COPD, but the symptoms are treatable and its progression can be delayed. The major goals of management are to reduce risk factors, manage stable COPD, prevent and treat acute exacerbations, and manage associated illnesses. The only measures that have been shown to reduce mortality are smoking cessation and supplemental oxygen. Stopping smoking decreases the risk of death by 18%. Other recommendations include influenza vaccination once a year, pneumococcal vaccination once every 5 years, and reduction in exposure to environmental air pollution. In those with advanced disease, palliative care may reduce symptoms, with morphine improving the feelings of shortness of breath. Noninvasive ventilation may be used to support breathing.

Pulmonary rehabilitation is a program of exercise, disease management and counseling, coordinated to benefit the individual. In those who have had a recent exacerbation, pulmonary rehabilitation appears to improve the overall quality of life and the ability to exercise, and reduce mortality. It has also been shown to improve the sense of control a person has over their disease, as well as their emotions. Breathing exercises in and of themselves appear to have a limited role. Pursed lip breathing exercises may be useful.

Being either underweight or overweight can affect the symptoms, degree of disability and prognosis of COPD. People with COPD who are underweight can improve their breathing muscle strength by increasing their calorie intake. When combined with regular exercise or a pulmonary rehabilitation program, this can lead to improvements in COPD symptoms. Supplemental nutrition may be useful in those who are malnourished.

Inhaled bronchodilators are the primary medications used and result in a small overall benefit. There are two major types, β₂ agonists and anticholinergics; both exist in long-acting and short-acting forms. They reduce shortness of breath, wheeze and exercise limitation, resulting in an improved quality of life. It is unclear if they change the progression of the underlying disease.

In those with mild disease, short-acting agents are recommended on an as needed basis. In those with more severe disease, long-acting agents are recommended. Long acting agents partly work by improving hyperinflation. If long-acting bronchodilators are insufficient, then inhaled corticosteroids are typically added. With respect to long-acting agents, it is unclear if tiotropium (a long-acting anticholinergic) or long-acting beta agonists (LABAs) are better, and it may be worth trying each and continuing the one that worked best. Both types of agent appear to reduce the risk of acute exacerbations by 15-25%. While both may be used at the same time, any benefit is of questionable significance.

There are several short-acting β₂ agonists available including salbutamol (Ventolin) and terbutaline. They provide some relief of symptoms for four to six hours. Long-acting β₂ agonists such as salmeterol and formoterol are often used as maintenance therapy. Some feel the evidence of benefits is limited while others view the evidence of benefit as established. Long-term use appears safe in COPD with adverse effects include shakiness and heart palpitations. When used with inhaled steroids they increase the risk of pneumonia. While steroids and LABAs may work better together, it is unclear if this slight benefit outweighs the increased risks.

There are two main anticholinergics used in COPD, ipratropium and tiotropium. Ipratropium is a short-acting agent while tiotropium is long-acting. Tiotropium is associated with a decrease in exacerbations and improved quality of life, and tiotropium provides those benefits better than ipratropium. It does not appear to affect mortality or the overall hospitalization rate. Anticholinergics can cause dry mouth and urinary tract symptoms. They are also associated with increased risk of heart disease and stroke. Aclidinium, another long acting agent which came to market in 2012, has been used as an alternative to tiotropium.

Corticosteroids are usually used in inhaled form but may also be used as tablets to treat and prevent acute exacerbations. While inhaled corticosteroids (ICS) have not shown benefit for people with mild COPD, they decrease acute exacerbations in those with either moderate or severe disease. By themselves they have no effect on overall one-year mortality. It is unclear if is they affect the progression of the disease. When used in combination with a LABA they may decrease mortality compared to either ICS or LABA alone. Inhaled steroids are associated with increased rates of pneumonia. Long-term treatment with steroid tablets is associated with significant side effects.

Long-term antibiotics, specifically those from the macrolide class such as erythromycin, reduce the frequency of exacerbations in those who have two or more a year. This practice may be cost effective in some areas of the world. Concerns include that of antibiotic resistance and hearing problems with azithromycin. Methylxanthines such as theophylline generally cause more harm than benefit and thus are usually not recommended, but may be used as a second-line agent in those not controlled by other measures. Mucolytics may help to reduce exacerbations in some people with chronic bronchitis. Cough medicines are not recommended.

Supplemental oxygen is recommended in those with low oxygen levels at rest (a partial pressure of oxygen of less than 50-55 mmHg or oxygen saturations of less than 88%). In this group of people it decreases the risk of heart failure and death if used 15 hours per day and may improve people's ability to exercise. In those with normal or mildly low oxygen levels, oxygen supplementation may improve shortness of breath. There is a risk of fires and little benefit when those on oxygen continue to smoke. In this situation some recommend against its use. During acute exacerbations, many require oxygen therapy; the use of high concentrations of oxygen without taking into account a person's oxygen saturations may lead to increased levels of carbon dioxide and worsened outcomes. In those at high risk of high carbon dioxide levels, oxygen saturations of 88-92% are recommended, while for those without this risk recommended levels are 94-98%.

For those with very severe disease, surgery is sometimes helpful and may include lung transplantation or lung volume reduction surgery. Lung volume reduction surgery involves removing the parts of the lung most damaged by emphysema allowing the remaining, relatively good lung to expand and work better. Lung transplantation is sometimes performed for very severe COPD, particularly in younger individuals.

Exacerbations. Acute exacerbations are typically treated by increasing the usage of short-acting bronchodilators. This commonly includes a combination of a short-acting inhaled beta agonist and anticholinergic. These medications can be given either via a metered-dose inhaler with a spacer or via a nebulizer with both appearing to be equally effective. Nebulization may be easier for those who are more unwell.

Oral corticosteroids improve the chance of recovery and decrease the overall duration of symptoms. They work equally well as intravenous steroids but appear to have fewer side effects. Five days of steroids work as well as ten or fourteen. In those with a severe exacerbation, antibiotics improve outcomes. A number of different antibiotics may be used including amoxicillin, doxycycline and azithromycin; it is unclear if one is better than the others. The FDA recommends against the use of fluoroquinolones when other options are available due to higher risks of serious side effects. There is no clear evidence for those with less severe cases.

For those with type 2 respiratory failure (acutely raised CO₂ levels) non-invasive positive pressure ventilation decreases the probability of death or the need of intensive care admission. Additionally, theophylline may have a role in those who do not respond to other measures.

Fewer than 20% of exacerbations require hospital admission. In those without acidosis from respiratory failure, home care (“hospital at home”) may be able to help avoid some admissions.

3. Bronchiolitis

Bronchiolitis is inflammation of the bronchioles, the smallest air passages of the lungs. It usually occurs in children less than two years of age with the majority being aged between three and six months. It presents with coughing, wheezing and shortness of breath which can cause some children difficulty in feeding. This inflammation is usually caused by respiratory syncytial virus (70% of cases) and is much more common in the winter months.

Treatment is typically supportive with oxygen, monitoring, fluid and nutrition perhaps by gastric tube or intravenously. The use of nebulized hypertonic saline is controversial with some reviews finding benefits and other not. There is insufficient evidence to support treatment with antibiotics, surfactant, chest physiotherapy or bronchodilators nebulized epinephrine. Bronchiolitis is common with up to one third of children being affected in their first year of life.

Signs and symptoms. In a typical case, an infant under two years of age develops cough, wheeze, and shortness of breath over one or two days. Crackles and/or wheeze are typical findings on listening to the chest with a stethoscope. The infant may be breathless for several days. After the acute illness, it is common for the airways to remain sensitive for several weeks, leading to recurrent cough and wheeze.

Some signs of severe disease include poor feeding (less than half of usual fluid intake in is preceding 24 hours), lethargy, history of apnea, respiratory rate>70/min, presence of nasal flaring and/or grunting, severe chest wall recession and cyanosis

Causes. The term usually refers to acute viral bronchiolitis, a common disease in infancy. This is most commonly caused by respiratory syncytial virus (RSV, also known as human pneumovirus). Other viruses which may cause this illness include metapneumovirus, influenza, parainfluenza, coronavirus, adenovirus, and rhinovirus.

Studies have shown there is a link between voluntary caesarean birth and an increased prevalence of bronchiolitis. A recent study by Perth's Telethon Institute for Child Health Research has shown an 11% increase in hospital admissions for children delivered this way. Children born prematurely (less than 35 weeks), with a low birth weight or who have from congenital heart disease may have higher rates of bronchiolitis and are more likely to require hospital admission. There is evidence that breastfeeding provides some protection against bronchiolitis.

Diagnosis. The diagnosis is typically made by clinical examination. Chest X-ray is sometimes useful to exclude bacterial pneumonia, but not indicated in routine cases.

Testing for the specific viral cause can be done but has little effect on management and thus is not routinely recommended. RSV testing by direct immunofluorescence testing on nasopharyngeal aspirate had a sensitivity of 61% and specificity of 89%. Identification of those who are RSV-positive can help for: disease surveillance, grouping (“cohorting”) people together in hospital wards to prevent cross infection, predicting whether the disease course has peaked yet, reducing the need for other diagnostic procedures (by providing confidence that a cause has been identified).

Infants with bronchiolitis between the age of two and three months have a second infection by bacteria (usually a urinary tract infection) less than 6% of the time. Preliminary studies have suggested that elevated procalcitonin levels may assist clinicians in determining the presence of bacterial coinfection, which could prevent unnecessary antibiotic use and costs.

Prevention. Prevention of bronchiolitis relies strongly on measures to reduce the spread of the viruses that cause respiratory infections (that is, handwashing, and avoiding exposure to those symptomatic with respiratory infections). In addition to good hygiene an improved immune system is a great tool for prevention. One way to improve the immune system is to feed the infant with breast milk, especially during the first month of life. Immunizations are available is for premature infants who meet certain criteria (some cardiac and respiratory disorders) such as

Palivizumab (a monoclonal antibody against RSV). Passive immunization therapy requires monthly injections every winter.

Management. Treatment of bronchiolitis is usually focused on the symptoms instead of the infection itself since the infection will run its course and complications are typically from the symptoms themselves. Without active treatment half of cases will go away in 13 days and 90% in three weeks.

High flow therapy with a high flow device that can provide precise flow/FiO₂ and medical grade vapor is an important part of the management of bronchiolitis.

Although inhaled epinephrine has been shown to decrease initial hospital admissions and overall length of stay compared to placebo in some studies, meta-analyses have found that it has no effect on the rate of one week post-emergency department visits or length of stay (although it does lead to shorter stay versus salbutamol). Nebulized and inhaled salbutamol (sold in the USA as Ventolin HFA) has been shown to decrease initial hospital admission rates in some studies, but no clear effect on length of stay in meta-analysis. The Society of Hospital Medicine recommends against routine use of these or other bronchodilators in children with bronchiolitis: “Published guidelines do not advocate the routine use of bronchodilators in patients with bronchiolitis. Comprehensive reviews of the literature have demonstrated that the use of bronchodilators in children admitted to the hospital with bronchiolitis has no effect on any important outcomes. There is limited demonstration of clear impact of bronchodilator therapy upon the course of disease. Additionally, providers should consider the potential impact of adverse events upon the patient.”

Nebulized hypertonic saline (3%) is controversial with some reviews finding benefit and others not.

D. Other Smooth Muscle Diseases

In addition to the aforementioned conditions, the methods and agent of the present disclosure may also be applied to other types of smooth muscle dysfunction such as vasospastic disorders, pulmonary hypertension, bladder and prostate dysfunction, chronic cramps, migraines, gastrointestinal and esophageal motility disorders, angina and multisystemic smooth muscle dysfunction syndrome .

### II. PROTEIN TYROSINE KINASE 2 BETA (PYK2)

Protein tyrosine kinase 2 beta is an enzyme that in humans is encoded by the PTK2B gene. This gene encodes a cytoplasmic protein tyrosine kinase that is involved in calcium-induced regulation of ion channels and activation of the map kinase signaling pathway. The encoded protein may represent an important signaling intermediate between neuropeptide-activated receptors or neurotransmitters that increase calcium flux and the downstream signals that regulate neuronal activity.

The encoded protein undergoes rapid tyrosine phosphorylation and activation in response to increases in the intracellular calcium concentration, nicotinic acetylcholine receptor activation, membrane depolarization, or protein kinase C activation.

This protein has been shown to bind a CRK-associated substrate, a nephrocystin, a GTPase regulator associated with FAK, and the SH2 domain of GRB2.

The encoded protein is a member of the FAK subfamily of protein tyrosine kinases but lacks significant sequence similarity to kinases from other subfamilies. Four transcript variants encoding two different isoforms have been found for this gene.

PTK2B has been shown to interact with BCAR1, Cb1 gene, DDEF2, DLG3, DLG4, Ewing sarcoma breakpoint region 1, FYN, GRIN2A, Gelsolin, NPHP1, PITPNM1, PTPN11, PTPN6, Paxillin, RAS p21 protein activator 1, RB1CC1, SORBS2, Src and TGFB1I1.

Inhibitors of PYK2 are disclosed in U.S. Patent Publications 20130281438, 20110281841, 20110269739, 20100256141, 20090318441, 20080234284 and 20080045561.

### III. ANAPLASTIC LYMPHOMA KINASE (ALK)

Anaplastic lymphoma kinase (ALK) also known as ALK tyrosine kinase receptor or CD246 (cluster of differentiation 246) is an enzyme that in humans is encoded by the ALK gene. ALK plays an important role in the development of the brain and exerts its effects on specific neurons in the nervous system.

The deduced amino acid sequences reveal that ALK is a novel receptor tyrosine kinase having a putative transmembrane domain and an extracellular domain. These sequences are absent in the product of the transforming NPM-ALK gene. ALK shows the greatest sequence similarity to LTK (leukocyte tyrosine kinase).

Pathology. The ALK gene can be oncogenic in three ways—by forming a fusion gene with any of several other genes, by gaining additional gene copies or with mutations of the actual DNA code for the gene itself.

The 2;5 chromosomal translocation is associated with approximately 60% anaplastic large-cell lymphomas (ALCLs). The translocation creates a fusion gene consisting of the ALK (anaplastic lymphoma kinase) gene and the nucleophosmin (NPM) gene: the 3′ half of ALK, derived from chromosome 2 and coding for the catalytic domain, is fused to the 5′ portion of NPM from chromosome 5. The product of the NPM-ALK fusion gene is oncogenic. In a smaller fraction of ALCL patients, the 3′ half of ALK is fused to the 5′ sequence of TPM3 gene, encoding for tropomyosin 3. In rare cases, ALK is fused to other 5′ fusion partners, such as TFG, ATIC, CLTC1, TPM4, MSN, ALO17, and MYH9.

The EML4-ALK fusion gene is responsible for approximately 3-5% of non-small-cell lung cancer (NSCLC). The vast majority of cases are adenocarcinomas. The standard test used to detect this gene in tumor samples is fluorescence in situ hybridization (FISH) by a US FDA approved kit. Recently Roche Ventana obtained approval in China and European Union countries to test this mutation by immunohistochemistry. Other techniques like reverse-transcriptase PCR (RT-PCR) can also be used to detect lung cancers with an ALK gene fusion but not recommended. ALK lung cancers are found in patients of all ages, although on average these patients tend to be younger. ALK lung cancers are more common in light cigarette smokers or nonsmokers, but a significant number of patients with this disease are current or former cigarette smokers. EML4-ALK-rearrangement in NSCLC is exclusive and not found in EGFR- or KRAS-mutated tumors.

Gene rearrangements and overexpression in other tumors include familial cases of neuroblastoma, inflammatory myofibroblastic tumor, adult and pediatric renal cell carcinomas, esophageal squamous cell carcinoma, breast cancer, notably the inflammatory subtype, colonic adenocarcinoma, glioblastoma multiforme and anaplastic thyroid cancer.

ALK inhibitors. Xalkori (crizotinib), produced by Pfizer, was approved by the FDA for treatment of late stage lung cancer on August 26, 2011. Early results of an initial Phase I trial with 82 patients with ALK induced lung cancer showed an overall response rate of 57%, a disease control rate at 8 weeks of 87% and progression free survival at 6 months of 72%. Ceritinib was approved by the FDA in April 2014 for the treatment of patients with anaplastic lymphoma kinase (ALK)-positive metastatic non-small cell lung cancer (NSCLC) who have progressed on or are intolerant to crizotinib. Alectinib (Chugai) is supported by an NDA filed in Japan, and has breakthrough status in U.S., with FDA approval in December of 2015.

Additional ALK inhibitors currently (or soon to be) undergoing clinical trials include Brigatinib (AP26113) (Ariad) (breakthrough status in U.S.) (also an EGFR inhibitor), Entrectinib (Nerviano's NMS-E628, licensed by Ignyta and renamed RXDX-101, in the U.S. orphan drug designation and rare pediatric disease designation for the treatment of neuroblastoma and orphan drug designation for treatment of TrkA-, TrkB-, TrkC-, ROS1- and ALK-positive NSCLC), PF-06463922 (Pfizer), TSR-011 (Tesaro), CEP-37440 (Teva) and X-396 (Xcovery).

### IV. TREATMENT

In accordance with the present disclosure, there are provided methods of treatment for airway smooth muscle dysfunction, some of which are mentioned above. The following discussion relates to pharmaceutical formulations and their use in such treatments.

A. Formulation and Administration

The present disclosure provides pharmaceutical compositions. Such compositions comprise a prophylactically or therapeutically effective amount of an agent, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a particular carrier when the pharmaceutical composition is administered intravenously. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium is chloride, dried skim milk, glycerol, propylene, glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulations can include standard carriers such as pharmaceutical grades of mannitol, lactose, starch, magnesium stearate, sodium saccharine, cellulose, magnesium carbonate, etc. Examples of suitable pharmaceutical agents are described in “Remington's Pharmaceutical Sciences.” Such compositions will contain a prophylactically or therapeutically effective amount of the antibody or fragment thereof, preferably in purified form, together with a suitable amount of carrier so as to provide the form for proper administration to the patient. The formulation should suit the mode of administration, which can be oral, intravenous, intra-arterial, intrabuccal, intranasal, nebulized, bronchial inhalation, or delivered by mechanical ventilation.

Pharmaceutical compositions of the present disclosure, as described herein, can be formulated for parenteral administration, e.g., formulated for injection via the intradermal, intravenous, intramuscular, subcutaneous, intra-tumoral or even intraperitoneal routes. The antibodies could alternatively be administered by a topical route directly to the mucosa, for example by nasal drops, inhalation, or by nebulizer. Pharmaceutically acceptable salts, include the acid salts and those which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Generally, the ingredients of compositions of the disclosure are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the disclosure can be formulated as neutral or salt forms. Pharmaceutically acceptable salts include those formed with anions such as those derived from is hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

B. Combination Therapies

In the context of the present disclosure, it also is contemplated that anti-PYK2/ALK compounds may be used in conjunction with “tradiational” airway smooth muscle disease therapies. To treat such diseases, using the methods and compositions of the present disclousre, one would generally administered the compounds according to the present disclosure in combination with at least one other agent. These compositions would be provided in a combined amount effective to treat airway smooth muscle disease. This process may involve administering both modalities at the same time. This may be achieved by using a single composition or pharmacological formulation that includes both agents, or by administering two distinct compositions or formulations, at the same time, wherein one composition includes the compounds of the present disclosure, and the other includes the other agent.

Alternatively, the compounds of the present disclosure may precede or follow the other treatment by intervals ranging from minutes to weeks. In embodiments where the modalities are applied separately to the cell, one would generally ensure that a significant period of time did not expire between the time of each delivery, such that the agent and expression construct would still be able to exert an advantageously combined effect on the target tissue. In such instances, it is contemplated that one would contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either the compounds of the present disclosure and other agent will be desired. Various combinations may be employed, where a compounds of the present disclosure therapy is “A” and the other therapy is “B”, as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are contemplated. Such “traditional” therapies include steroid-based therapies, leukotriene modifying therapies, bronchodilatory therapies or beta-agonist therapies.

### V. EXAMPLES

The following examples are included to demonstrate particular embodiments. It should be appreciated by those of skill in the art that the techniques disclosed in the examples that follow represent techniques discovered by the inventors to function well in the practice of embodiments, and thus can be considered to constitute particular modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### Materials and Methods

Animals. The inventors previously reported production of CFTR^{-/-} and CFTR^{-/-} pigs (27). Animal studies were reviewed and approved by the University of Iowa Animal Care and Use Committee.

Airway smooth muscle tissue and cells. Trachea from newborn WT (CFTR^{+/-} and CFTR^{+/-}) or CFTR^{-/-} pigs was excised and placed in modified Krebs Solution containing (mM): 120.8 NaCl, 5.9 KCl, 0.2 CaCl₂, 1.2 MgCl₂, 1.2 NaH₂PO₄, 2 NaHCO₃, 11 d-glucose, and 10 HEPES, pH 7.4. The trachealis muscle was dissected by removing the cartilage and epithelial layers. For force measurements, strips of trachealis smooth muscle were hung in a tissue perfusion system (Radnoti LLC, Monrovia, Calif.). For cell isolation, the remaining medial layers were minced and digested using a papain dissociation system (Worthington, Lakewood, N.J.) based upon previous studies (10). The minced smooth muscle was suspended in Earle's balanced salt solution (EBSS) containing papain (10 U/ml) and DNase (1000 U/ml) and incubated at 37° C. for 1 hr. Collagenase (0.5mg/m1) was added and after 1 hr the mixture triturated. Primary cells were grown to confluence in a 1:1 mixture of DMEM and Ham's F12 on fibrinogen-coated 6-well plates. All solutions contained 100 U/ml penicillin and 0.1 mg/ml streptomycin. Primary culture cells were allowed to grow for 8-10 d until confluent. Positive immunostaining with the polyclonal anti-α-smooth muscle actin antibody (clone 1A4, abcam) confirmed the presence of airway smooth muscle cells and lack of staining with the monoclonal anti-fibroblast surface protein antibody (clone 1B10, abcam) excluded the presence of fibroblasts.

Measurement of airway smooth muscle force generation. Prior to airway smooth muscle isolation, freshly harvested tracheal rings were bubbled in ringer's solution overnight in the presence of either 10 μM NVP or DMSO vechile. The next day, airway smooth muscle strips (1.5 mm×3 mm) from the trachealis muscle were mounted along the trachealis muscle orientation in individual muscle baths (Radnoti Glass, Monrovia, Calif.) filled with 28 ml carbogenated physiological buffer solution (in mmol/L; 120 NaCl, 25 NaHCO₃, 11 glucose, 1.8 CaCl₂, 4.7 KCl, 1.2 MgSO₄, 1.2 KH₂PO₄) at 37° C. The contractile activity was recorded with Grass isometric force transducers and amplifiers connected to a Biopac data acquisition system (Biopac Systems, Goleta, Calif.). The muscle strips were equilibrated in the muscle bath and length was adjusted until tension was registered in the calibrated force transducer. The muscle strips remained at this reference length for 60 min at 37° C. before they were tested for contractile response to acetylcholine. The smooth muscle force from NVP-TAE684 or DMSO pretreated strips was determined by obtaining concentration-response curves to acetylcholine (10⁻⁹ to 10⁻² M) in the muscle bath. The strips were allowed to equilibrate before testing differing concentrations of acetylcholine.

Precision cut lung slice preparation. Precision cut lung slices were isolated as previously described (11) from wild type newborn pigs. Lung slice culture media consisted of Dulbecco's Modified Eagle Medium (Gibco, Big Cabin, Okla.), 10% fetal bovine serum (Gibco, Big Cabin, Okla.), 1% penicillin-streptomycin (5,000 U/mL, Gibco, Big Cabin, Okla.), and 0.1% gentamicin (50 mg/mL, Gibco, Big Cabin, Okla.).

For the porcine lung slice studies, the tracheal bronchus from collected tracheal lobes was cannulated with a 24 G IV catheter (BD Medical) and a tight seal was created using suture. The tracheal lobe was instilled with approximately 10 mL of 2% UltraPure Low Melting Point agarose (Invitrogen, Carlsbad, Calif.) solution and cooled at −20° C. for 30 min. After cooling, a lung tissue block was isolated with the tracheal lobe caudal airway branch located centrally. The lung tissue block was mounted to a compresstome (VF-300, Precisionary Instruments, Greenville, N.C.) and the tissue block was surrounded using 2% Type 1-B, low EEO agarose (Sigma-Aldrich, St. Louis, Mo.). 300 μm slices were prepared and slice quality was ensured by assessing for a complete epithelial layer surrounding the lumen, visualization of beating cilia, and no major tears or holes in alveolar and parenchymal tissues surrounding the airway lumen. A custom-made perfusion chamber was created for experimental protocols and microscopic imaging of lung slices (IX-81 Olympus, 4× magnification, recording at 2 fps, 37° C., 5% CO₂ chamber). For luminal diameter changes in response to NVP-TAE684, lung slices were pretreated overnight with either NVP-TAE684 (10 μM, Selleck Chemical, Houston, Tex.) or DMSO (0.1% final concentration) and then mounted in the perfusion chamber and perfused with HBSS for 5 minutes. Lung slices were perfused with increasing concentrations of methacholine (0-10 μM, Sigma-Aldrich, St. Louis, Mo.) to induce airway contraction in the lung slice. For all PCLS experiments, maximal airway contraction was analyzed for each dose by lumen area measurements performed in ImageJ (NIH). Paired comparisons were performed using sequentially cut lung slices pretreat with either NVP-TAE684 or DMSO.

Measurement of in vivo airway resistance. WT mice were pretreated with either PYK2 inhibitor NVP-TAE684 (100 μl of 5 mM, 1% DMSO) or vehicle (1% DMSO) was given via intra-peritoneal injections daily for 3 days prior and including the day of pulmonary measurements. Mice were anesthetized with ketamine (50 or 100 mg/kg) and xylazine (0-10 mg/kg). Following tracheotomy, an 18-gauge cannula was inserted and securely tied with 4.0 braided silk. The animals were then mechanically ventilated with a computer-controlled small-animal ventilator (FlexiVent, SCIREQ, Montreal, Quebec) using a tidal volume of 8 mL/kg at a rate of 180 breaths/min and PEEP of 3.5 cm H₂O. Pulmonary resistance was measured by the computer controlled ventilator by interrupting ventilation and imposing broadband low-frequency oscillatory waveforms and then resuming ventilation. After measuring baseline resistance, mice underwent airway challenge with normal saline and increasing dosages of methacholine by aerosol challenge of 20 μl of solutions ranging from 0.3 to 100 mg/ml. Measurements of resistance were obtained for each dose at every ten seconds for 2 minutes and then the next methacholine dose was administered.

Statistical analysis. All dose-response curves were analyzed using a four-parameter logistic regression algorithm. In all figures, the NVP-TAE684 group was statistically different is from the control-DMSO group based upon this analysis (p<0.05).

### Example 2

### Results

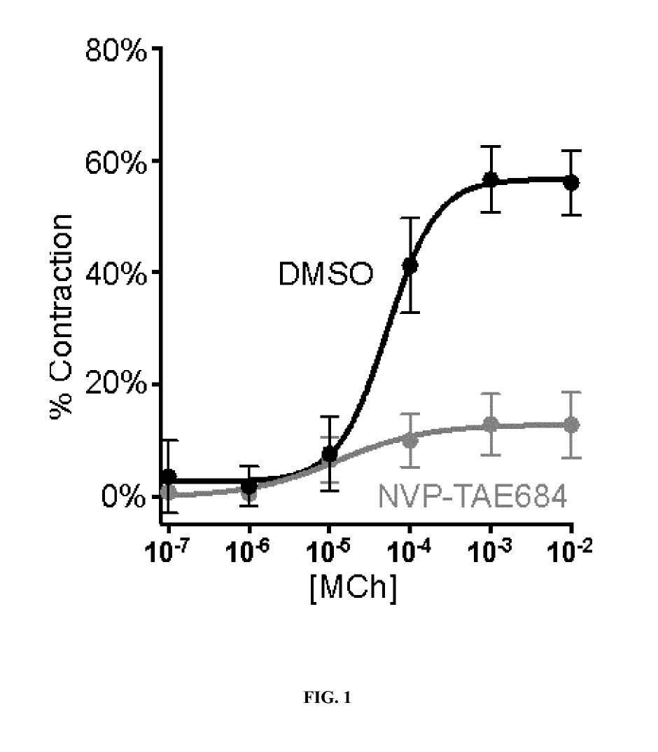

NVP-TAE684 inhibits cholinergic induced airway smooth muscle contraction. To investigate whether NVP-TAE684 has anti-airway hyper-responsive properties, as predicted by the LINCS database, lung slices from WT newborn pigs were cut and the cross-sectional area of the airway lumen was measured. The luminal area from WT pigs pretreated with DMSO decreased following perfusion of increasing dosages of methacholine (0-1 mM) (FIG. 1). A parallel experiment was conducted in WT lung slices pretreated with NVP-TEA684. Pretreatment with NVP-TAE684 decreased the airway narrowing of WT lung slices in response to increasing dosages of methacholine as measured by luminal area (FIG. 1). These results indicate that NVP-TAE684 can decrease cholinergic induced airway contractility in porcine lung slices.

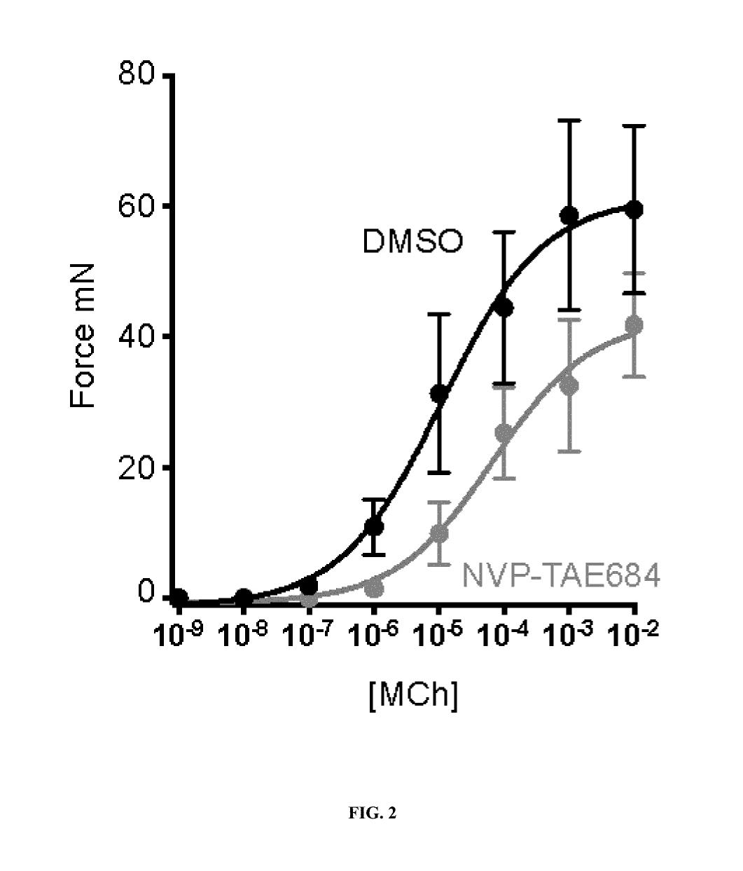

Given that precision cut lung slices possess numerous cell types including cartilage, epithelia, and smooth muscle (11), the inventors hypothesized that the decreased response of airways in lung slices to methacholine was, in part, due to a direct effect on airway smooth muscle. To test this hypothesis, the inventors isolated tracheal airway smooth muscle strips from newborn WT pigs. Tracheal muscle strips pretreated with DMSO were contracted using increasing doses of acetylcholine and exhibited a dose response reaching a maximum force production of approximately 60 mN (FIG. 2). An identical experiment was also performed on tracheal muscle strip that had been pretreated with NVP-TAE684. NVP-TAE684 treatment decreased force generation in tracheal smooth muscle strips across all doses of acetylcholine and diminished the maximal force production (40 mN) (FIG. 2). There were no significant differences in the median effective concentrations of acetylcholine between the two experiments. These results suggest that NVP-TAE684 prevents cholinergic induced airway hyper-responsiveness.

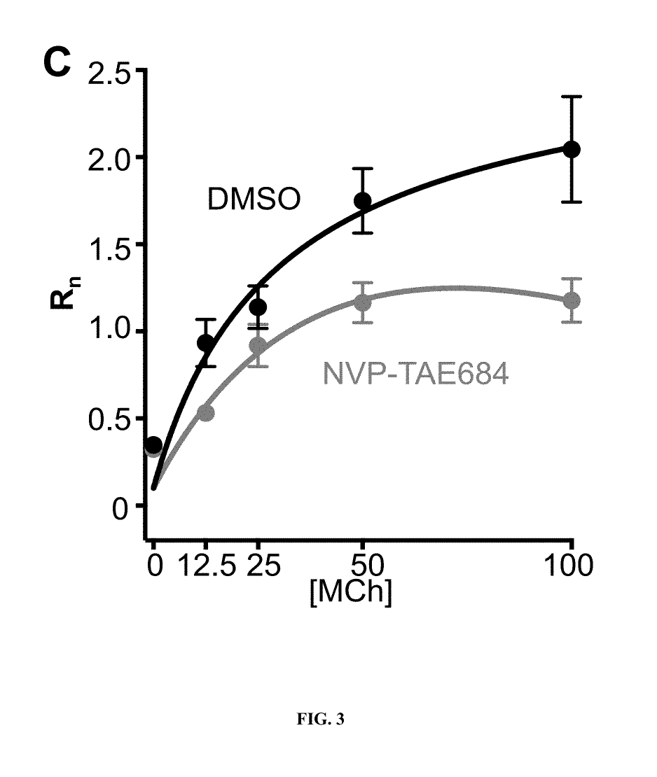

NVP-TAE684 and Ceritinib inhibit cholinergic induced airway hyper-responsiveness in vivo. The inventors evaluated the effect of NVP-TAE684 on pulmonary reactivity to methacholine in wild-type mice in vivo. NVP-TAE684 (100 μl of 5 mM, 1% DMSO) or vehicle (1% DMSO) was given via intrapeitoneal injections daily for 3 days prior and including the day of pulmonary measurements. The respiratory response to inhaled methacholine of wild-type mice receiving NVP-TAE684 was significantly decreased compared to that of wild-type mice receiving the vehicle control alone (FIG. 3). Morphologic analysis of histological sections taken from the lung parenchyma, conducting airways, and trachea demonstrated that there were no significant changes in epithelia, cartilage or airway smooth muscle layers in NVP-TAE684 treated mice as compared to vehicle control mice. Additionally, no evidence of drug-induced toxicity was present in histological sections of heart, kidney, and liver. These findings suggest that 1) NVP-TAE684 can prevent cholinergic induced airway reactivity in vivo and 2) the LINCS approach to novel compound discovery using a porcine model can identify targets cross-species.

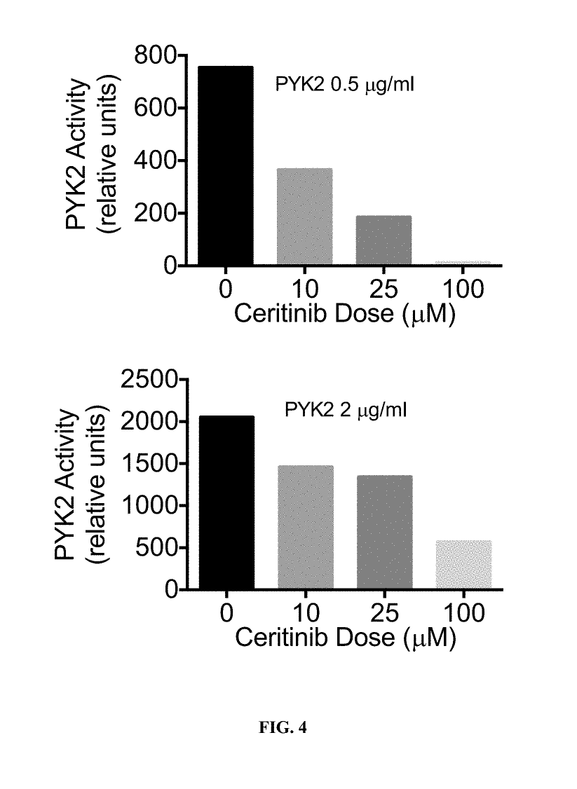

Similar to the effects of NVP-TAE684 on PY2K activity, the inventors also found that ceritinib caused a dose-dependent decrease in PYK-2 activity (FIG. 4). These findings suggest that both NVP-TAE684 and ceritinib can inhibit PYK-2.

### Example 3

### Discussion

The inventors' previous studies have shown that loss of CFTR contributes to an enhanced basal airway smooth muscle tone (5) and suggested that loss of CFTR in airway smooth muscle may lead to changes in airway reactivity. They performed a whole transcriptome assessment of airway smooth muscle from CF and WT newborn pigs. The inventors' primary findings are that 1) that there is a subset of genes dysregulated in airway smooth muscle in response to CFTR knockout, 2) these genes represent changes in muscle contraction, cellular proliferation, migration, and global cell signaling processes, 3) the CF transcriptome along with connectivity mapping can be used to investigate airway smooth muscle biology as well as generate possible therapeutic targets, and 4) NVP-TAE684 may represent a therapeutic agent for diseases of airway narrowing.

Connectivity mapping and subsequent phosphorylation studies demonstrated that PYK2, a protein tyrosine kinase, is over active in CF airway smooth muscle and could be inhibited to reduce airway smooth muscle tone. Interestingly, PYK2 inhibition has been shown to reduce airway hyper-responsiveness in ovalbumin-sensitized mice (12), however this study did not explore smooth muscle effects of PYK2 inhibition.

Using an integrative approach combining genetically modified pigs, next generation transcriptional sequencing, physiological assays, in vivo assays, and molecular biology, the is inventors have identified a novel therapeutic role for NVP-TAE648 in diseases of airway narrowing and airway hyper-responsiveness.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VI. REFERENCES

The following references, to the extent that they provide exemplary procedural or other details supplementary to those set forth herein, are specifically incorporated herein by reference.
Berridge et al., Nat Rev Mol Cell Biol, 1:11-21, 2000.
Cook et al., Am J Respir Crit Care Med, 193 :417-426, 2016.
Mitchell et al., J Pediatr, 93:744-748, 1978.
Pascoe et al., Can J Physiol Pharmacol, 93:137-143, 2015.
Remington's Pharmaceutical Sciences” 15th Edition, chapter 33, in particular pages 624-652.
van Haren et al., Eur Respir J, 4:301-307, 1991.
van Haren et al., Eur Respir J, 5:1083-1088, 1992.
Weinberger, Clin Rev Allergy Immunol 23:77-85, 2002.
Woodruff, Proceedings of the American Thoracic Society, 5:113-118, 2008.
Yick et al., Allergy, 69(9):1233-40, 2014.
U.S. Patent Publication 20130281438
U.S. Patent Publication 20110281841
U.S. Patent Publication 20110269739
U.S. Patent Publication 20100256141
U.S. Patent Publication 20090318441
U.S. Patent Publication 20080234284
U.S. Patent Publication 20080045561

What is claimed is:

1. A method of treating airway smooth muscle dysfunction in a subject comprising administering to said subject an agent selected from compound having the formula: 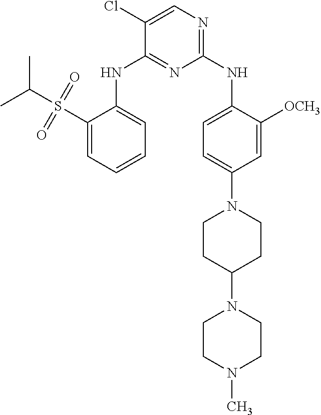 [SMILES: COc1cc(N2CCC(N3CCN(C)CC3)CC2)ccc1Nc1ncc(Cl)c(Nc2ccccc2S(=O)(=O)C(C)C)n1]
or a derivative thereof, or Certinib, or a derivative thereof.

2. The method of claim 1, wherein said agent is Ceretinib or a derivative thereof.

3. The method of 1, wherein said agent is compound having the formula: 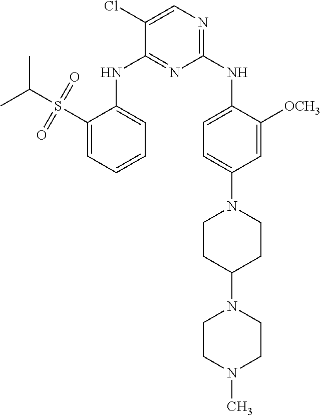 [SMILES: COc1cc(N2CCC(N3CCN(C)CC3)CC2)ccc1Nc1ncc(Cl)c(Nc2ccccc2S(=O)(=O)C(C)C)n1]
or a derivative thereof.

4. The method of claim 1, wherein said smooth muscle dysfunction is airway narrowing.

5. The method of claim 4, wherein said airway narrowing is asthma or chronic obstructive pulmonary disease.

6. The method of claim 5, wherein said asthma is cystic fibrosis-induced asthma.

7. The method of claim 1, wherein administering comprises systemic administration.

8. The method of claim 7, wherein administering comprises oral administration or intravenous administration.

9. The method of claim 1, wherein administering comprises loco-regional administration.

10. The method of claim 9, wherein loco-regional administration comprises inhalation administration.

11. The method of claim 1, wherein said subject is a human.

12. The method of claim 1, wherein said subject is a non-human mammal.

13. The method of claim 12, wherein said non-human mammal is a pig.

14. The method of claim 1, further comprising treating said subject with a second therapy that improves airway function.

15. The method of claim 14, wherein said second therapy is a steroid-based therapy, a leukotriene modifying therapy, a bronchodilatory therapy or a beta-agonist therapy.

16. The method of claim 14, wherein said second therapy is administered at the same time as said compound.

17. The method of claim 14, wherein said second therapy is administered before or after said compound.

18. The method of claim 1, wherein said compound is administered more than once.

19. The method of claim 1, wherein said compound is administered daily, every other day, every third day, bi-weekly, every fourth day, weekly, every two weeks, or monthly.

20. The method of claim 1, wherein said compound is administered on a chronic basis.

21. A method of treating smooth muscle dysfunction in a subject comprising administering to said subject an agent that inhibits tyrosine protein kinase 2 beta activity, wherein the smooth muscle dysfunction is not asthma.

22. The method of claim 21, wherein administering comprises systemic administration.

23. The method of claim 22, wherein administering comprises oral administration or intravenous administration.

24. The method of claim 21, wherein administering comprises loco-regional administration.

25. The method of claim 21, wherein said subject is a human.

26. The method of claim 21, wherein said subject is a non-human mammal.

27. The method of claim 26, wherein said non-human mammal is a pig.

28. The method of claim 21, wherein said agent is administered more than once.

29. The method of claim 21, wherein said agent is administered daily, every other day, every third day, bi-weekly, every fourth day, weekly, every two weeks, or monthly.

30. The method of claim 21, wherein said agent is administered on a chronic basis.

31. The method of claim 21, wherein the smooth muscle dysfunction is selected from the group consisting of vasospastic disorders, pulmonary hypertension, bladder and prostate dysfunction, chronic cramps, migraines, gastrointestinal and esophageal motility disorders, and angina.

32. The method of claim 21, wherein the agent is selected from compound having the formula: 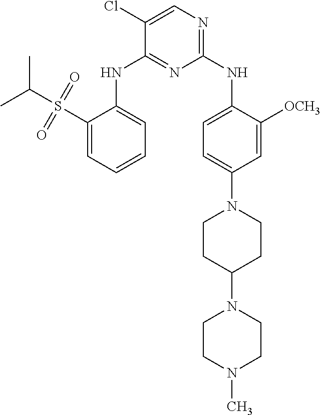 [SMILES: COc1cc(N2CCC(N3CCN(C)CC3)CC2)ccc1Nc1ncc(Cl)c(Nc2ccccc2S(=O)(=O)C(C)C)n1]
or a derivative thereof, or Certinib, or a derivative thereof.
